(19) United States
(12) Patent Application Publication
(10) Pub. No.: US 2025/0092156 A1
(43) Pub. Date: Mar. 20, 2025

(54) DE-CLUSTERING INHIBITORS OF PTPRA

(71) Applicant: The Regents of the University of California, Oakland, CA (US)

(72) Inventor: Nunzio Bottini, Beverly Hills, CA (US)

(21) Appl. No.: 18/290,730

(22) PCT Filed: Jul. 29, 2022
(86) PCT No.: PCT/US2022/074340

§ 371 Date: Jan. 19, 2024

Related U.S. Application Data
(60) Provisional application No. 63/228,083, filed on Jul. 31, 2021.

(51) Int. Cl. C07K 16/40; A61K 39/00; A61P 35/00

(52) U.S. Cl. CPC C07K 16/40; A61P 35/00; A61K 2039/505

(57) ABSTRACT

Described herein are methods and compositions to treat arthritis, fibrosis and cancer by inhibiting RPTPα clustering. Included are methods and compositions for the diagnosis of RPTPα clustering.

### CROSS REFERENCE TO RELATED APPLICATIONS

This application claims priority under 35 U.S.C. § 119 from Provisional Application Ser. No. 63/228,083, filed Jul. 31, 2021, the disclosures of which are incorporated herein by reference in their entirety.

### TECHNICAL FIELD

Described herein are methods and compositions to treat arthritis, fibrosis and cancer by inhibiting RPTPα clustering. Also included are methods and compositions for the diagnosis of RPTPα clustering.

### INCORPORATION BY REFERENCE OF SEQUENCE LISTING

Accompanying this filing is a Sequence Listing entitled, “00015-405WO1.xml” created on Jan. 29, 2022 and having 23, 301 bytes of data, machine formatted on IBM-PC, MS-Windows operating system using WIPO Standard ST. 26 formatting. The sequence listing is hereby incorporated by reference in its entirety for all purposes.

### BACKGROUND

Protein tyrosine phosphatases (PTPs) regulate a wide variety of signal transduction processes by counterbalancing the action of protein tyrosine kinases. The human genome encodes 22 putative transmembrane PTPs, classified in 8 subtypes. The R4 subtype receptor-type protein phosphatase α (RPTPα) is an example of transmembrane PTP with two intracellular catalytic domains. The membrane proximal domain called D1 is active in substrate dephosphorylation, while the membrane distal domain called D2 displays very limited catalytic activity in vitro but is involved in functional regulation of the D1 domain. The membrane proximal catalytic domain (D1 domain) of RPTPα can dimerize in vitro through a trans interaction between a wedge motif of one monomer and the active site of the other monomer (Bilwes, A M, et al. 1996 Nature 382:555-559; Jiang, G, et al. 1999 Nature 401:606-610). The extracellular domain of RPTPα is short and becomes glycosylated (Daum, G et al. 1994. J Biol Chem 269 (14): 10524-8).

### SUMMARY

Recruitment and activation of SRC by receptor-type protein phosphatase x (RPTPα, also called RPTPA) in fibroblasts plays a role in fibrosis and arthritis. Until now an assessment of RPTPα clustering in primary cells has not been demonstrated. This disclosure describes RPTPα clustering through enhanced resolution microscopy-based and analytical imaging techniques in primary fibroblast-like synoviocytes (FLS). Super-resolution microscopy of migrating FLS demonstrated proximity of RPTPα with other RPTPα and with SRC molecules in the context of actin stress fibers. The P210L/P211L mutation in the wedge region of RPTPα that impairs RPTPα dimerization reduced RPTPα-RPTPα localization, however, it also unexpectedly reduced localization proximity of RPTPα and SRC in these cells. Additional types of analyses confirmed this unexpected result, leading to the present disclosure that clustering of RPTPα in FLS and other fibroblasts has a stimulatory role rather than an inhibitory role on the function of RPTP. This is opposite to what has been proposed in the literature.

The disclosure shows that incubation of FLS with a monoclonal antibody (Mab-2F8) developed against the extracellular domain of RPTPα results in RPTPα-dependent inhibition of cell migration. The inhibition of cell migration correlated with Mab-2F8-mediated de-clustering of RPTPα and a reduced association between RPTPα and SRC in both sensitized emission (SE) and acceptor photobleaching (AP) fluorescence resonance energy transfer (FRET) microscopy-based assays.

The disclosure surprisingly demonstrates that in FLS cell types relevant to arthritis and fibrosis and in cancer, clustering leads to activation of RPTPα and agents that decluster RPTPα can be used as therapeutics for these diseases. The data show that de-clustering rather than cross-linking of PTPRα is needed in order to accomplish functional inhibition for arthritis and fibrosis. Furthermore, the reduction in association with SRC demonstrates that declustering agents will have utility in the treatment of cancer.

The disclosure provides a method of treating an autoimmune disease in a subject in need thereof, the method comprising administering to said subject an effective amount of a protein tyrosine phosphatase receptor type A (RPTPα) antagonist to a subject. In one embodiment, the RPTPα antagonist reduces the invasiveness or migration of the subject's fibroblast-like synoviocytes. In another embodiment, the RPTPα antagonist is an anti-RPTPα antibody. In a further embodiment, the anti-RPTPα antibody binds an extracellular portion of RPTPα. In still another embodiment, the anti-RPTPα antibody is an anti-RPTPα dimer inhibiting antibody or an anti-RPTPα declustering antibody. In still another embodiment of any of the foregoing embodiments, the autoimmune disease is arthritis or a fibroblast mediated disease. In a further embodiment, the arthritis is rheumatoid arthritis, psoriatic arthritis, juvenile idiopathic arthritis, or osteoarthritis. In yet another embodiment of any of the foregoing embodiments, the autoimmune disease is selected from the group consisting of multiple sclerosis, systemic lupus erythematosus (SLE), myasthenia gravis, juvenile onset diabetes, diabetes mellitus type 1, Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, psoriasis, ichthyosis, Graves ophthalmopathy, inflammatory bowel disease, Addison's disease, Vitiligo, asthma, scleroderma, systemic sclerosis, and allergic asthma. In still another or further embodiment, the subject comprises fibroblast-like synoviocytes comprising clustered RPTPα and increased RPTPα activity relative to a standard control.

The disclosure also provides a method of treating a disease in a subject, the method comprising administering to the subject a therapeutically effective amount of a recombinant polypeptide, wherein administration treats the disease in the subject, wherein the recombinant polypeptide causes de-clustering of RPTPα, and wherein the disease is selected from the group consisting of an autoimmune disease, an inflammatory autoimmune disease, a fibroblast-mediated disease, or cancer. In one embodiment, the autoimmune disease is arthritis. In another embodiment, the autoimmune disease is rheumatoid arthritis. In still another embodiment, the subject has a fibroblast-mediated disease. In a further embodiment, the fibroblast-mediated disease is fibrosis. In a further embodiment, the fibrosis is selected from the group consisting of pulmonary fibrosis, idiopathic pulmonary fibrosis, liver fibrosis, endomyocardial fibrosis, atrial fibrosis, mediastinal fibrosis, myelofibrosis, retroperitoneal fibrosis, nephrogenic systemic fibrosis, skin fibrosis, and arthrofibrosis. In another embodiment, the disease is cancer.

The disclosure also provides an antibody which binds to one or more epitopes which has or have an amino acid sequence present in SEQ ID NO: 1 or SEQ ID NO: 2 or which comprises the amino acid sequence of SEQ ID NO: 1 or SEQ ID NO: 2. In another embodiment, the disclosure provides the antibody in a pharmaceutically acceptable carrier.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1A shows the number density of clustered RPTPα in the leading edge (LEDGE) or actin-rich stress fibers (BSTRESS) of wild-type (WT) RPTPα-expressing fibroblast-like synoviocytes (FLS) with separation of co-localized molecules grouped at <65 nm vs >65 nm. Each point represents a transfected cell and data was pooled from multiple experiments *** p≤0.001, ** p≤0.01, * p≤0.05 by Mann-Whitney test.

FIG. 1B shows the number density of clustered RPTPα-SRC in LEDGE or BSTRESS actin-rich stress fibers of WT RPTPα-expressing FLS with separation of co-localized molecules grouped at <65 nm vs >65 nm. Each point represents a transfected cell and data was pooled from multiple experiments *** p≤0.001, ** p≤0.01, * p≤0.05 by Mann-Whitney test.

FIG. 1C shows the relative density of clustered (<65 nm distance) RPTPα in LEDGE and BSTRESS actin-rich stress fibers of RPTPα knockout (KO) cells expressing FLAG-tagged WT RPTPα and P210L/P211L mutant (P210) RPTP. Each point represents a transfected cell and data was pooled from four independent experiments using four different RPTPα KO cell lines for WT, three independent experiments using three different RPTPα KO cell lines for P210. * * p≤0.001, ** p≤0.01, * p≤0.05 by Mann-Whitney test.

FIG. 1D shows the relative density of clustered (<65 nm distance) RPTPα-SRC in LEDGE and BSTRESS actin-rich stress fibers of RPTPα KO cells expressing WT and P210 RPTPα. Each point represents a transfected cell and data was pooled from four independent experiments using four different RPTPα KO cell lines for WT, three independent experiments using three different RPTPα KO cell lines for P210. *** p≤0.001, ** p≤0.01, * p≤0.05 by Mann-Whitney test.

FIG. 2A shows sensitized emission confocal FRET (SECFRET) signal quantitatively outlined and scored (upper panel) and calculated % FRET efficiency (lower panel) of RPTPα homodimer in the leading edge of RPTPα KO FLS transfected with plasmids to express both HA-tagged and FLAG-tagged WT RPTPα (WT-WT), P210 mutant RPTPα (P210-P210) , or delta D2 domain mutant RPTPα (dD2-dD2) RPTPα. Each point represents a transfected cell and data were pooled from three independent experiments using three different RPTPα KO cell lines. **** p≤0.0001, *** p≤0.001, * p≤0.05 by Mann-Whitney test.

FIG. 2B shows SECFRET signal quantitatively outlined and scored (upper panel) and calculated & FRET efficiency (lower panel) of RPTPα-SRC association in the leading edge of RPTPα KO FLS transfected with plasmids to express FLAG-tagged WT, P210, or dD2 RPTPα. Each point represents a transfected cell and data were pooled from three independent experiments using three different RPTPα KO cell lines. **** p≤0.0001, *** p≤0.001, * p≤0.05 by Mann-Whitney test.

FIG. 3A shows post-bleaching fluorescence intensity and % FRET efficiency of donor in spectral acceptor photobleach (SapFRET) assay of RPTPα clustering in RPTPα KO FLS transfected to express WT-WT, P210-P210 and dD2-dD2 RPTPα. Each point represents a region of interest which stringently outlines the cell lamellipodium. N=5-10 cells per construct from 2 experiments using 2 RPTPa KO lines. **** p≤0.0001, *** p≤0.001, ** p≤0.01, by Kluskal-Wallis or Mann-Whitney on the area under the curve (AUC).

FIG. 3B shows post-bleaching fluorescence intensity and % FRET efficiency of donor in SapFRET assay of RPTPα-SRC association in RPTPα KO FLS transfected to express WT RPTPα with SRC (WT-SRC), P210 RPTPα with SRC (P210-SRC) and dD2 RPTPα with SRC (dD2-SRC). Each point represents a region of interest which stringently outlines the cell lamellipodium. N=5-10 cells per construct from 2 experiments using 2 RPTPα KO lines. **** p≤0.0001, *** p≤0.001, ** p≤0.01, by Kluskal-Wallis or Mann-Whitney on the AUC.

FIG. 3C shows quantification of % FLIM-FRET efficiency of clustered RPTPα in RPTPα KO FLS transfected to express WT RPTPα with SRC (WT-SRC), P210 RPTPα with SRC (P210-SRC) and dD2 RPTPα with SRC (dD2-SRC). Each point represents a transfected cell. N=20-60 cells per construct across 2 experiments using 2 RPTPα KO lines **** p≤0.0001, *** p≤0.001, ** p≤0.01, by Kluskal-Wallis or Kluskal-Wallis or Mann-Whitney on the AUC.

FIG. 4A shows quantification of RPTPα positive area, SRC positive area and cortactin positive area in the LEDGE of migrating RPTPα KO FLS transfected to express WT, P210, or dD2 RPTPα. Each point represents a transfected cell and data were pooled from five independent experiments using different RPTPα KO cell lines. **** p≤0.0001, *** p≤0.001, ** p≤0.01, * p≤0.05 by Kluskal-Wallis test.

FIG. 4B shows quantification of colocalization by Mander's overlap coefficient analysis of RPTPα-SRC, colocalized RPTPα-cortactin and colocalized SRC-cortactin in the LEDGE of migrating RPTPα KO FLS transfected to express WT, P210, or dD2 RPTPα. Each point represents a transfected cell and data were pooled from five independent experiments using different RPTPα KO cell lines. **** p≤0.0001, *** p≤0.001, ** p≤0.01, * p≤0.05 by Kluskal-Wallis test.

FIG. 5A shows diagrams to depict the experimental setting for the following panels of FIG. 5. RPTPα KO FLS were transfected to express FLAG-tagged WT, P210, or dD2 RPTPα.

FIG. 5B shows representative images of migrated FLS transfected with empty vector (EV) or plasmids encoding WT, P210 or dD2 RPTPα, 24 hours after seeding in a transwell migration assay.

FIG. 5C shows migration rate of FLS transfected with EV or plasmids encoding WT, P210 or dD2 RPTPα, normalized by the migration of the EV condition in each experiment. Graph shows means and SEM and each point represents a different RPTPα KO cell line. **** p≤0.0001, *** p≤0.001, ** p≤0.01, * p≤0.05 by Kluskal-Wallis test.

FIG. 5D shows representative images of scratched wound area in monolayers of FLS transfected with EV or plasmids encoding WT, P210 or dD2 RPTPα, 24 hours after wounding.

FIG. 5E shows quantification of wound area reduction (wound closure) in monolayers of FLS transfected with EV or plasmids encoding WT, P210 or dD2 RPTPα normalized by the wound area of the EV condition in each experiment. Graph shows means and SEM of the ratio between the area after 24 hours and the area at time 0 normalized by the area change of the EV condition in each experiment. Each point represents a different αKO cell line. **** p<0.0001, *** p≤0.001, ** p≤0.01, * p≤0.05 by Kluskal-Wallis test.

FIG. 5F shows phosphorylation of SRC on Y416 and Y527 in FLS transfected with EV or plasmids encoding WT, P210 or dD2 RPTPα. The left panel shows representative Western blots of lysates. The right panel shows quantification of the densitometric phospho-SRC Y416/SRC signal ratio. Graph shows means and SEM of the ratio relative to the EV condition in each experiment and each point represents a different αKO cell line. **** p≤0.0001, *** p≤0.001, ** p≤0.01, * p≤0.05 by Kluskal-Wallis test.

FIG. 6 shows Western blotting image of lysates of FLS from KO or B6 FLS immunoblotted with anti-RPTPα Ab.

FIG. 7A shows drawn schematic of the experimental targets and reagents labelling strategy for the experiments shown in the following panels of FIG. 7. RPTPα KO FLS were transfected with FLAG-tagged WT RPTPα with (panels B, C, WT-WT) or without (panels D, E, WT-SRC) HA-tagged WT RPTP expression constructs and incubated with or without anti-RPTPα Ab.

FIG. 7B shows quantification of total SECFRET signal areas of RPTPα homodimers at the LEDGE of migrating FLS. Each point represents a transfected cell and data were pooled from two independent experiments using different RPTPα KO FLS lines. **** p≤0.0001, *** p≤0.001, ** p≤0.01, * p≤0.05 by Mann-Whitney test.

FIG. 7C shows post-bleaching time resolved average fluorescence intensity profile of dequenched donor signal of RPTPα clustering using the SapFRET method in WT-WT FLS incubated with or without anti-RPTPα Ab. Each point represents a transfected cell and data were pooled from two independent experiments using different RPTPα KO FLS lines. **** p≤0.0001, *** p≤0.001, ** p≤0.01, * p≤0.05 by Mann-Whitney test on the AUC.

FIG. 7D shows quantification of total SECFRET signal areas at the LEDGE of WT-SRC FLS incubated with or without anti-RPTPα Ab. Each point represents a transfected cell and data were pooled from two independent experiments using different RPTPα KO FLS lines. **** p≤0.0001, *** p≤0.001, ** p≤0.01, * p≤0.05 by Mann-Whitney test.

FIG. 7E shows representative images of migrated FLS from C57BL/6 (B6) or RPTPα KO (KO) FLS incubated with anti-RPTPα Ab or isotype control Ab, 24 h after seeding.

FIG. 7F shows migration rate of B6 or KO FLS, normalized by migration of FLS incubated with isotype control Ab. Graphs show means and SEM and each point represents a different RPTPα KO cell line. **** p≤0.0001, *** p≤0.001, ** p≤0.01, * p≤0.05 by Mann-Whitney test.

FIG. 7G shows representative images of scratched wound area in monolayers of B6 or KO FLS incubated with anti-RPTPα Ab or isotype control Ab, 24 h after wounding.

FIG. 7H shows wound area reduction in monolayers of B6 or KO FLS incubated with anti-RPTPα Ab or isotype control Ab. Graph shows means and SEM of the ratio between the area at 24 h and the area at time 0 normalized by the area change of FLS incubated with control Ab in each experiment and each point represents a different FLS line. **** p≤0.0001, *** p≤0.001, ** p≤0.01, * p≤0.05 by Mann-Whitney test.

### DETAILED DESCRIPTION

As used herein and in the appended claims, the singular forms “a,” “an,” and “the” include plural referents unless the context clearly dictates otherwise. Thus, for example, reference to “a cell” includes a plurality of such cells and reference to “the agent” includes reference to one or more agents, and so forth.

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Any publications discussed above and throughout the text are provided solely for their disclosure prior to the filing date of the present application. Nothing herein is to be construed as an admission that the inventor is not entitled to antedate such disclosure by virtue of prior disclosure.

A key function of RPTPα is to activate the kinase SRC via dephosphorylation of its inhibitory Tyr527 residue. Activation of SRC by RPTPα involves formation of an RPTPα-SRC complex, and interaction between the SH2 domain of SRC and a phosphorylated Tyr789 (Tyr825 in the isoform used in this work) residue in the D2 domain of RPTPα; although it remains to be clarified whether that is the only mechanism.

RPTPα is overexpressed in several cancers and has been long considered a drug target to reduce activation of SRC and cancer cell growth. RPTPα also plays an important role in the pathogenic action of fibroblast populations. It has been shown that RPTPα enhances transforming growth factor beta (TGFB)-mediated myofibroblast formation and collagen deposition, and RPTPα deletion ameliorates disease severity in models of pulmonary fibrosis. In inflammatory arthritis, RPTPα is highly expressed in a local joint-lining fibroblast population called fibroblast-like synoviocytes (FLS) and promotes the pathogenic action of these cells by enhancing SRC-mediated FLS migration and responsiveness to proinflammatory cytokines. Accordingly, deletion of RPTPα protects mice from arthritis in an FLS-dependent mouse model.

Pharmacological modulation of protein tyrosine phosphatases (PTPs) through allosteric inhibitors has reignited the interest in PTPs as potential drug targets but also highlighted the need to better understand specific regulation mechanisms of each PTP in order to design appropriate targeting strategies. RPTPα is regulated by dimerization wherein the D1 domain can form a symmetric dimer through an interaction between a juxtamembrane wedge motif (encompassing aa 211-213) of one monomer and the active site of the other monomer. Dimeric RPTPα exists in two different states whose balance depends on the intracellular D2 domain. It has been postulated that oxidation of the D2 domain leads dimeric RPTPα to assume a “rotated” conformation characterized by a different reciprocal topology of the two monomers, and linked to inhibition of RPTPα function and SRC activation.

The disclosure provides data regarding dimerization in intact primary fibroblasts using enhanced resolution microscopy-based and analytical imaging techniques. The studies used migrating primary FLS because much of the pathogenic action of FLS in rheumatoid arthritis (RA) is due to excessive migration, and it has been shown that knockdown or deletion of RPTPα is able to impair this FLS phenotype. Utilization of sensitized emission (SE) FRET, acceptor photobleaching (AP) FRET, and fluorescence lifetime imaging (FLIM) microscopy based analytical methodologies in conjunction with Nikon stochastic optical reconstruction microscopy (N-STORM) localization super resolution microscopy defined, quantified and characterized spatial and compartmental localization of RPTPα molecules with other RPTPα and with SRC molecules. These results were complemented with functional assays of FLS expressing known dimerization-impairing RPTPα mutants.

Results showed that RPTPα undergoes substantial polarized clustering on the surface of migrating FLS, but they also unexpectedly showed that in FLS, RPTPα clustering promotes the recruitment of SRC and the function of RPTPα. The disclosure provides evidence that the model of dimerization-induced inhibition of RPTPα might not be universally valid across all cell types and RPTPα functions. Moreover, the disclosure demonstrates that modulators of RPTPα activity which reduce clustering, i.e. declustering agents, are useful in treating FLS-mediated disorders, such as rheumatoid arthritis (RA), and other disorders in which RPTPα promotes disease, such as fibrosis and cancer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, gamma-carboxyglutamate, and 0-phosphoserine.

Amino acid analogs refers to compounds that have the same basic chemical structure as a naturally occurring amino acid, i.e., an x carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody will be most critical in specificity and affinity of binding. Antibodies or fragments of antibodies may be derived from different organisms, including humans, mice, rats, hamsters, camels, etc. Antibodies disclosed herein may include antibodies that have been modified or mutated at one or more amino acid positions to improve or modulate a desired function of the antibody (e.g., glycosylation, expression, antigen recognition, effector functions, antigen binding, specificity).

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{HI} by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

For preparation of suitable antibodies as disclosed herein and for use according to the methods disclosed herein, e.g., recombinant, monoclonal, or polyclonal antibodies, many techniques known in the art can be used (see, e.g., Kohler & Milstein, Nature 256:495-497 (1975); Kozbor et al., Immunology Today 4:72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3. sup.rd ed. 1997)).

Techniques for the production of single chain antibodies or recombinant antibodies (U.S. Pat. Nos. 4,946,778, 4,816,567) can be adapted to produce antibodies to polypeptides as disclosed herein. Also, transgenic mice, or other organisms such as other mammals, may be used to express humanized or human antibodies (see, e.g., U.S. Pat. Nos. 5,545,807; 5, 545, 806; 5, 569, 825; 5,625,126; 5, 633, 425; 5, 661,016, Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg & Huszar, Intern. Rev. Immunol. 13:65-93 (1995)). Alternatively, phage display technology can be used to identify antibodies and heteromeric Fab fragments that specifically bind to selected antigens (see, e.g., Mccafferty et al., Nature 348:552-554 (1990); Marks et al., Biotechnology 10:779-783 (1992)). Antibodies can also be made bispecific, i.e., able to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or immunotoxins (see, e.g., U.S. Pat. No. 4,676,980, WO 91/00360; WO 92/200373; and EP 03089).

Methods for humanizing or primatizing non-human antibodies are well known in the art (e.g., U.S. Pat. Nos. 4,816,567; 5,530,101; 5,859,205; 5,585,089; 5,693,761; 5,693,762; 5,777,085; 6,180,370; 6,210,671; and 6,329,511; WO 87/02671; EP Patent Application 0173494; Jones et al. (1986) Nature 321:522; and Verhoyen et al. (1988) Science 239:1534). Humanized antibodies are further described in, e.g., Winter and Milstein (1991) Nature 349:293. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers (see, e.g., Morrison et al., PNAS USA, 81:6851-6855 (1984), Jones et al., Nature 321:522-525 (1986); Riechmann et al., Nature 332:323-327 (1988); Morrison and Oi, Adv. Immunol., 44:65-92 (1988), Verhoeven et al., Science 239:1534-1536 (1988) and Presta, Curr. Op. Struct. Biol. 2:593-596 (1992), Padlan, Molec. Immun., 28:489-498 (1991); Padlan, Molec. Immun., 31 (3): 169-217 (1994)), by substituting rodent complement determining regions (CDRs) or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some framework (FR) residues are substituted by residues from analogous sites in rodent antibodies. For example, polynucleotides comprising a first sequence coding for humanized immunoglobulin framework regions and a second sequence set coding for the desired immunoglobulin complementarity determining regions can be produced synthetically or by combining appropriate CDNA and genomic DNA segments. Human constant region DNA sequences can be isolated in accordance with well-known procedures from a variety of human cells.

The term “antibody fragment” refers to at least one portion of an antibody, that retains the ability to specifically interact with (e.g., by binding, steric hindrance, stabilizing/destabilizing, spatial distribution) an epitope of an antigen. Examples of antibody fragments include, but are not limited to, Fab, Fab′, F(ab′h, Fv fragments, scFv antibody fragments, disulfide-linked Fvs (sdFv), a Fd fragment consisting of the VH and CHl domains, linear antibodies, single domain antibodies such as sdAb (either vL or vH), camelid vHH domains, multi-specific antibodies formed from antibody fragments such as a bivalent fragment comprising two Fab fragments linked by a disulfide bridge at the hinge region, and an isolated CDR or other epitope binding fragments of an antibody. An antigen binding fragment can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23:1126-1136, 2005). Antigen binding fragments can also be grafted into scaffolds based on polypeptides such as a fibronectin type III (Fn3) (see U.S. Pat. No. 6,703,199, which describes fibronectin polypeptide mini bodies).

The term “antibody heavy chain,” refers to the larger of the two types of polypeptide chains present in antibody molecules in their naturally occurring conformations, and which normally determines the class to which the antibody belongs.

The term “antibody light chain,” refers to the smaller of the two types of polypeptide chains present in antibody molecules in their naturally occurring conformations. Kappa (K) and lambda (A) light chains refer to the two major antibody light chain isotypes.

“Biological sample” or “sample” refer to materials obtained from or derived from a subject or patient. A biological sample includes sections of tissues such as biopsy and autopsy samples, and frozen sections taken for histological purposes. Such samples include bodily fluids such as blood and blood fractions or products (e.g., serum, plasma, platelets, red blood cells), sputum, tissue, cultured cells (e.g., primary cultures, explants, and transformed cells) stool, urine, synovial fluid, joint tissue, synovial tissue, synoviocytes, fibroblast-like synoviocytes, macrophage-like synoviocytes, immune cells, hematopoietic cells, fibroblasts, macrophages, T cells, etc. A biological sample is typically obtained from a eukaryotic organism, such as a mammal such as a primate e.g., chimpanzee or human; cow; dog; cat; a rodent, e.g., guinea pig, rat, mouse; rabbit; or a bird; reptile; or fish.

The terms “anti-RPTPA antibody” and ‘anti-RPTPα antibody” refer to an antibody directed to RPTPα or antibody fragment or non-immunoglobulin anti-RPTPA protein that selectively binds to an RPTPA protein or fragment thereof.

An “autoimmune disease therapeutic agent”, an “inflammatory autoimmune disease (IAD) therapeutic agent”, a “fibrotic disease therapeutic agent” or a “cancer therapeutic agent” is a molecule (e.g. RPTPα binding agent, antibody, peptide, ligand mimetic, small chemical molecule) that treats or prevents the indicated disease (i.e. autoimmune, inflammatory autoimmune, fibrotic, cancer) when administered to a subject in a therapeutically effective dose or amount. In embodiments, any one of these therapeutic agents is an RPTPα binding agent.

A “biopsy” refers to the process of removing a tissue sample for diagnostic or prognostic evaluation, and to the tissue specimen itself Any biopsy technique known in the art can be applied to the diagnostic and prognostic methods disclosed herein. The biopsy technique applied will depend on the tissue type to be evaluated (i.e., prostate, lymph node, liver, bone marrow, blood cell, joint tissue, synovial tissue, synoviocytes, fibroblast-like synoviocytes, macrophage-like synoviocytes, immune cells, hematopoietic cells, fibroblasts, macrophages, T cells, etc.), the size and type of a tumor (i.e., solid or suspended (i.e., blood or ascites)), among other factors. Representative biopsy techniques include excisional biopsy, incisional biopsy, needle biopsy, surgical biopsy, and bone marrow biopsy. Biopsy techniques are discussed, for example, in Harrison's Principles of Internal Medicine, Kasper, et al., eds., 16th ed., 2005, Chapter 70, and throughout Part V.

As used herein, the term “CDR” or “complementarity determining region” is intended to mean the non-contiguous antigen binding sites found within the variable region of both heavy and light chain polypeptides. These particular regions have been described by Kabat et al., J. Bioi. Chern. 252:6609-6616 (1977); Kabat et al., U.S. Dept. of Health and Human Services, “Sequences of proteins of immunological interest” (1991); Chothia et al., J. Mol. Bioi. 196:901-917 (1987); and MacCallum et al., J. Mol. Bioi. 25 262:732-745 (1996), where the definitions include overlapping or subsets of amino acid residues when compared against each other. Nevertheless, application of either definition to refer to a CDR of an antibody or grafted antibodies or variants thereof is intended to be within the scope of the term as defined and used herein. As used herein, the different CDRs of an antibody could be also defined by a combination of the different definitions. For example, vHCDR1 could be defined based on Kabat and VHCDR2 could be defined based on Chothia. The amino acid residues which encompass the CDRs as defined by each of the above cited references are as follows:

CDR DEFINITIONS

| | Kabat | Chothia | MacCallum |
|---|---|---|---|
| VHCDR1 | 31-35 | 26-32 | 30-35 |
| VHCDR2 | 50-65 | 53-55 | 47-58 |
| VHCDR3 | 95-102 | 96-10 | 193-101 |
| VLCDR1 | 24-34 | 26-32 | 30-36 |
| VLCDR2 | 50-56 | 50-52 | 46-55 |
| VLCDR3 | 89-97 | 91-96 | 89-96 |

(Residue Numbers correspond to the identified reference) .

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. The preferred antibodies of, and for use according to the present disclosure include humanized and/or chimeric monoclonal antibodies.

As used herein “clustering” of RPTPα refers to the association of multiple monomers of RPTPα, dimerization or oligomerization of RPTPα.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles disclosed herein.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine(S), Threonine (T); and 8) Cysteine (C), Methionine (M) (see, e.g., Creighton, Proteins (1984)).

The term “diagnosis” refers to a relative probability that a disease (e.g., disease related to arthritis or fibrosis, an autoimmune disease, an inflammatory autoimmune disease, or cancer) is present in the subject. The term “prognosis” refers to a relative probability that a certain future outcome may occur in the subject with respect to a disease state. For example, in the present context, prognosis can refer to the likelihood that an individual will develop a disease (e.g., a disease related to arthritis or fibrosis, an autoimmune disease, an inflammatory autoimmune disease, or cancer), or the likely severity of the disease (e.g., extent of pathological effect and duration of disease). The terms are not intended to be absolute, as will be appreciated by any one of skill in the field of medical diagnostics.

The terms “dose” and “dosage” are used interchangeably herein. A dose refers to the amount of active ingredient given to an individual at each administration, or to an amount administered in vitro or ex vivo. For the methods and compositions provided herein, the dose may generally depend on the required treatment for the disease (e.g., an autoimmune disease, an inflammatory autoimmune disease, a fibrotic disease or cancer), and the biological activity of the RPTPα binding agent, RPTPα antagonist, anti-RPTPα antibody, or RPTPA ligand mimetic. The dose will vary depending on a number of factors, including the range of normal doses for a given therapy, frequency of administration; size and tolerance of the individual; severity of the condition; risk of side effects; and the route of administration. One of skill will recognize that the dose can be modified depending on the above factors or based on therapeutic progress. The term “dosage form” refers to the particular format of the pharmaceutical or pharmaceutical composition, and depends on the route of administration. For example, a dosage form can be in a liquid form for nebulization, e.g., for inhalants, in a tablet or liquid, e.g., for oral delivery, or a saline solution, e.g., for injection.

By “effective amount,” “therapeutically effective amount,” “therapeutically effective dose or amount” and the like as used herein is meant an amount (e.g., a dose) that produces effects for which it is administered (e.g., treating or preventing a disease). The exact dose and formulation will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Remington: The Science and Practice of Pharmacy, 20th Edition, Gennaro, Editor (2003), and Pickar, Dosage Calculations (1999)). For example, for the given parameter, a therapeutically effective amount will show an increase or decrease of at least 58, 108, 158, 20%, 258, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a standard control. A therapeutically effective dose or amount may ameliorate one or more symptoms of a disease. A therapeutically effective dose or amount may prevent or delay the onset of a disease or one or more symptoms of a disease when the effect for which it is being administered is to treat a person who is at risk of developing the disease.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

A “label” or a “detectable moiety” is a composition detectable by spectroscopic, photochemical, biochemical, immunochemical, radiochemical, chemical, or other physical means. For example, useful labels include 32P, fluorescent dyes, electron-dense reagents, enzymes (e.g., as commonly used in an ELISA), biotin, digoxigenin, or haptens and proteins or other entities which can be made detectable, e.g., by incorporating a radiolabel into a peptide or antibody specifically reactive with a target peptide. Any method known in the art for conjugating an antibody to the label may be employed, e.g., using methods described in Hermanson, BIOCONJUGATE TECHNIQUES 1996, Academic Press, Inc., San Diego.

As used herein, the term “pharmaceutically acceptable” is used synonymously with “physiologically acceptable” and “pharmacologically acceptable”. A pharmaceutical composition will generally include agents for buffering and preservation in storage, and can include buffers and carriers for appropriate delivery, depending on the route of administration.

“Pharmaceutically acceptable excipient” and “pharmaceutically acceptable carrier” refer to a substance that aids the administration of an active agent to and/or absorption by a subject and can be included in the compositions disclosed herein without causing a significant adverse toxicological effect on the patient. Unless indicated to the contrary, the terms “active agent,” “active ingredient,” “therapeutically active agent,” “therapeutic agent” and like are used synonymously. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, polyethylene glycol, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds disclosed herein. One of skill in the art will recognize that other pharmaceutical excipients are useful in the methods and compositions disclosed herein.

Certain compounds disclosed herein can exist in unsolvated forms as well as solvated forms, including hydrated forms. In general, the solvated forms are equivalent to unsolvated forms and are intended to be encompassed within the scope of the present disclosure. Certain compounds disclosed herein may exist in multiple crystalline or amorphous forms. In general, all physical forms are equivalent for the uses contemplated herein and are intended to be within the scope of the present disclosure.

The term “polynucleotide”, “nucleic acid”, or “recombinant nucleic acid” refers to polymers of nucleotides such as deoxyribonucleic acid (DNA), and, where appropriate, ribonucleic acid (RNA).

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

A “protein level of RPTPα” refers to an amount (relative or absolute) of RPTPα in its protein form (as distinguished from its precursor RNA form). A protein of RPTPα may include a full-length protein (e.g., the protein translated from the complete coding region of the gene, which may also include post-translational modifications), functional fragments of the full-length protein (e.g., sub-domains of the full-length protein that possess an activity or function in an assay), or protein fragments of RPTPα, which may be any peptide or oligopeptide of the full-length protein.

The terms “RPTPα”, “RPTPA”, “RPTPα”, “PTPRA”, and “PTPa”, are used interchangeably herein, and refer to receptor tyrosine-protein phosphatase alpha. It is understood that the term “RPTPα” in the context of a gene refers to the gene encoding receptor tyrosine-protein phosphatase alpha. In certain embodiments, RPTPα means the full length RPTPα (e.g., the protein translated from the complete coding region of the gene, which may also include post-translational modifications). In certain other embodiments RPTPα includes a fragment of the RPTPα full length protein or a functional fragment of the full length RPTPα protein. In some embodiments this definition includes one or all splice variants of an RPTPα. An RPTPα may include all homologs of the RPTPα. In other embodiments, RPTPα refers to mammalian RPTPα. In still other embodiments, RPTPα refers to a human RPTPα. In still other embodiments, RPTPα includes all splice variants of the RPTPα (e.g., 1, 2, 3, 4, 5, 6, 7, 8, 9, 10, or more splice variants).

The term “RPTPα” as provided herein includes any of the receptor-type tyrosine-protein phosphatase alpha (RPTPα) naturally occurring forms, homologs or variants that maintain the phosphatase activity (e.g., within at least 508, 808, 908, 95%, 968, 978, 98%, 99% or 100% activity compared to the native protein). In some embodiments, variants have at least 908, 958, 968, 978, 988, 99% or 100% amino acid sequence identity across the whole sequence or a portion of the sequence (e.g. a 50, 100, 150 or 200 continuous amino acid portion) compared to a naturally occurring form. In some embodiments, the RPTPα protein is the protein as identified by the NCBI sequence reference GI: 4506303. In other embodiments, the RPTPα protein is encoded by a nucleic acid sequence identified by the NCBI sequence reference GI: 125987583. In one embodiment, human PTPRα has the coding sequence of SEQ ID NO: 13 and the polypeptide sequence of SEQ ID NO:15.

The terms “RPTPA antagonist” and “RPTPα antagonist” refer to an agent which interferes with the function of RPTPα. The phrase “interferes with the function of RPTPα” includes, but is not limited to, one or more of the following: declustering of RPTPα, reducing the colocalization of RPTPα with SRC, reduction in the level of enzymatic activity of RPTPα, and reduction in the level of expression (e.g. through reduced nucleic acid or protein production) of RPTPα. In some embodiments, an RPTPα antagonist may interfere with one or more RPTPα functions directly. In other embodiments, an RPTPα antagonist may interfere with one or more RPTPα functions indirectly. An RPTPα antagonist can be an RPTPα binding agent, an RPTPα small molecule inhibitor, an RPTPα allosteric inhibitor, an anti-RPTPα antibody, or an RPTPα ligand mimetic, as disclosed herein. In one embodiment, an RPTPα antagonist is the extracellular domain of RPTPα. In certain embodiments, the extracellular domain of RPTPα comprises SEQ ID NO: 10, SEQ ID NO: 11, or SEQ ID NO: 12.

An “RPTPα binding agent” is a molecule that binds (e.g. preferentially binds) to RPTPα. Where the molecule preferentially binds, the binding is preferential as compared to other macromolecular biomolecules present in an organism or cell. A compound preferentially binds to as compared to other macromolecular biomolecules present in an organism or cell, for example, when the preferential binding is 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, 2000-fold, 3000-fold, 4000-fold, 5000-fold, 6000-fold, 7000-fold, 8000-fold, 9000-fold, 10000 fold, 100,000-fold, 1,000,000-fold greater. In embodiments, the RPTP binding agent is a protein, peptide, RPTPα extracellular domain, ligand, ligand mimetic, or a small chemical molecule. In embodiments, an RPTPα binding agent disrupts the interaction between RPTPα and a physiological or natural ligand. In embodiments, an RPTPα binding agent binds a physiological or natural ligand of RPTPα. In embodiments, an RPTPα binding agent binds the complex of RPTPα bound to a ligand.

An “RPTPα ligand mimetic” is an RPTPα binding agent that is designed to mimic, in structure or in binding mode, a known RPTPα ligand or is capable of inhibiting the binding of a natural or physiological ligand to RPTPα. In embodiments, an RPTPα ligand mimetic is a synthetic chemical compound, peptide, protein, fusion protein (e.g., RPTPα-Fc), peptidomimetic, or modified natural ligand. For example, an RPTPα ligand mimetic may bind the same amino acids or a subset of the same amino acids on RPTPα that a natural ligand of RPTPα binds during the physiological functioning of RPTPα. RPTPα ligand mimetics include biopolymers (e.g., proteins, nucleic acids, or sugars), lipids, chemical molecules with molecular weights less than five hundred (500) Daltons, one thousand (1000) Daltons, five thousand (5000) Daltons, less than ten thousand (10,000) Daltons, less than twenty five thousand (25,000) Daltons, less than fifty thousand (50,000) Daltons, less than seventy five thousand (75,000), less than one hundred thousand (100,000), or less than two hundred fifty thousand (250,000) Daltons. In embodiments, the synthetic chemical compound is greater than two hundred fifty thousand (250,000) Daltons. In certain embodiments, the RPTPα binding agent is less than five hundred (500) Daltons.

In embodiments, an RPTPα ligand mimetic is a small chemical molecule. The term “small chemical molecule” and the like, as used herein, refers to a molecule that has a molecular weight of less than two thousand (2000) Daltons. In embodiments, a small chemical molecule is a molecule that has a molecular weight of less than one thousand (1000) Daltons. In other embodiments, a small chemical molecule is a molecule that has a molecular weight of less than five hundred (500) Daltons. In other embodiments, a small chemical molecule is a molecule that has a molecular weight of less than five hundred (500) Daltons.

In other embodiments, a small chemical molecule is a molecule that has a molecular weight of less than one hundred (100) Daltons.

Any of the therapeutic agents of this invention may “target” RPTPα, by binding (e.g. preferentially binding) to RPTPα. Where preferentially binding, the agent binds preferentially to a targeted molecule compared to its binding to other molecules of a similar form (e.g., other RPTPs). An agent preferentially binds to a molecule, for example, when the binding to the targeted molecule is greater than the binding to other molecules of a similar form. In embodiments, the preferential binding is 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, 2000-fold, 3000-fold, 4000-fold, 5000-fold, 6000-fold, 7000-fold, 8000-fold, 9000-fold, 10000 fold, 100,000-fold, 1,000,000-fold greater.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

The term “scFv” refers to a fusion protein comprising at least one antibody fragment comprising a variable region of a light chain and at least one antibody fragment comprising a variable region of a heavy chain, wherein the light and heavy chain variable regions are contiguously linked, e.g., via a synthetic linker, e.g., a short flexible polypeptide linker, and capable of being expressed as a single chain polypeptide, and wherein the scFv retains the specificity of the intact antibody from which it is derived. Unless specified, as used herein an scFv may have the vL and vH variable regions in either order, e.g., with respect to the N-terminal and C-terminal ends of the polypeptide, the scFv may comprise vL-linker-vH or may comprise vH-linker-vL

The term “SRC” as provided herein includes any of sarcoma tyrosine kinase (SRC) naturally occurring forms, homologs or variants that maintain the kinase activity (e.g., within at least 50%, 808, 90%, 958, 968, 978, 98%, 99% or 100% activity compared to the native protein). In some embodiments, variants have at least 90%, 95%, 96%, 978, 98%, 99% or 100% amino acid sequence identity across the whole sequence or a portion of the sequence (e.g. a 50, 100, 150 or 200 continuous amino acid portion) compared to a naturally occurring form. In some embodiments, the SRC protein is the protein as identified by the NCBI sequence reference GI: 4885609. In other embodiments, the SRC protein is encoded by a nucleic acid sequence identified by the NCBI sequence reference GI: 520262038. In one embodiment, human SRC has the coding sequence of SEQ ID NO: 14 and the polypeptide sequence of SEQ ID NO: 16.

The phrase “specifically (or selectively) binds” or “specifically (or selectively) immunoreactive with,” when referring to a protein or peptide, refers to a binding reaction that is determinative of the presence of a protein, often in a heterogeneous population of proteins and other biologics. Thus, under designated immunoassay conditions, an antibody binds to a particular protein at least two times the background and more typically more than 10 to 100 times background. Specific binding to an antibody under such conditions requires an antibody that is selected for its specificity for a particular protein. For example, polyclonal antibodies can be selected to obtain only those polyclonal antibodies that are specifically immunoreactive with a selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998) for a description of immunoassay formats and conditions that can be used to determine specific immunoreactivity).

A “standard control” refers to a sample, measurement, or value that serves as a reference, usually a known reference, for comparison to a test sample, measurement, or value. For example, a test sample can be taken from a patient suspected of having a given disease (e.g., an autoimmune disease, inflammatory autoimmune disease, cancer, infectious disease, immune disease, or other disease) and compared to a known normal (i.e., non-diseased) individual (e.g., a standard control subject). A standard control can also represent an average measurement or value gathered from a population of similar individuals (e.g., standard control subjects) that do not have a given disease (e.g., standard control population), of healthy individuals with a similar medical background, same age, weight, etc. A standard control value can also be obtained from the same individual, e.g., from an earlier-obtained sample from the patient prior to disease onset. One of skill will recognize that standard controls can be designed for assessment of any number of parameters (e.g., RNA levels, protein levels, individual RPTP levels, specific cell types, specific bodily fluids, specific tissues, synoviocytes, synovial fluid, synovial tissue, fibroblast-like synoviocytes, macrophage-like synoviocytes, skin and lung fibroblasts).

One of skill in the art will understand which standard controls are most appropriate in a given situation and be able to analyze data based on comparisons to standard control values. Standard controls are also valuable for determining the significance (e.g., statistical significance) of data, as known in the art.

The terms “subject,” “patient,” “individual,” etc. are not intended to be limiting and can be generally interchanged. That is, an individual described as a “patient” does not necessarily have a given disease, but may be merely seeking medical advice.

A “test agent” as provided herein may be a nucleic acid, peptide, antibody or small molecule. In some embodiments, the test agent is a nucleic acid. In other embodiments, the test agent is a peptide. In still other embodiments, the test agent is a polypeptide (e.g., an antibody). In some embodiments, the test agent is a small molecule.

As used herein, the terms “treat” and “prevent” may refer to any delay in onset, reduction in the frequency or severity of symptoms, amelioration of symptoms, reduction in risk of developing symptoms, improvement in patient comfort or function (e.g., joint function), decrease in severity of the disease state, etc. The effect of treatment can be compared to an individual or pool of individuals not receiving a given treatment, or to the same patient prior to, or after cessation of, treatment. The term “prevent” generally refers to a decrease in the occurrence of a given disease (e.g., diseases related to arthritis or fibrosis, autoimmune disease, inflammatory autoimmune disease, and cancer) or disease symptoms in a patient. As indicated above, the prevention may be complete (no detectable symptoms) or partial, such that fewer symptoms are observed than would likely occur absent treatment.

In a first embodiment, the disclosure provides a method of treating an autoimmune disease in a subject in need thereof, the method including administering to the subject an effective amount of an RPTPα antagonist. In certain embodiments, the autoimmune disease is a fibroblast mediated disease, arthritis, osteoarthritis, rheumatoid arthritis, psoriatic arthritis, juvenile idiopathic arthritis, multiple sclerosis, systemic lupus erythematosus (SLE), myasthenia gravis, juvenile onset diabetes, diabetes mellitus type 1, Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, psoriasis, ichthyosis, Graves ophthalmopathy, inflammatory bowel disease, Addison's disease, Vitiligo, asthma, scleroderma, systemic sclerosis, or allergic asthma. In other embodiments, the autoimmune disease is arthritis. In still other embodiments, the autoimmune disease is rheumatoid arthritis. In another embodiment, the autoimmune disease is psoriatic arthritis. In yet another embodiment, the disease is non-autoimmune arthritis. In still another embodiment, the non-autoimmune arthritis is osteoarthritis. In another embodiment, the disease is a fibrotic disease. In yet another embodiment, the fibrotic disease includes idiopathic pulmonary fibrosis, fibrotic lung diseases, scleroderma, liver fibrosis, liver sclerosis, and/or advanced glomerulonephritis, nephrosclerosis.

The disclosure also provides a method of treating cancer in a subject in need thereof the method including administering to the subject an effective amount of an RPTPα antagonist. In one embodiment, the cancer is lung (small cell and non-small cell), thyroid, prostate, pancreatic, breast, ovarian or colon, sarcoma or melanoma. In another embodiment, the cancer is of the blood, brain, leukemia, chronic lymphocytic leukemia, chronic myeloid leukemia, colorectal, gastrointestinal stromal tumor, kidney, lymphoma, or multiple myeloma.

The disclosure also provides a method of decreasing inflammation in a synovium of a subject in need thereof, the method including administering to the subject an effective amount of an RPTPA antagonist.

In various embodiments of the disclosure comprising treating an autoimmune disease or decreasing inflammation in a synovium, the subject presents with fibroblast-like synoviocytes that express high levels of RPTPα relative to a standard control as disclosed herein. In certain embodiments, the subject has rheumatoid arthritis.

In certain embodiments, treating an autoimmune disease, a fibrotic disease, cancer, or decreasing inflammation further includes decreasing TNF activity, PDGF activity and/or IL-1 activity in FLS. In one embodiment, the method includes decreasing TNF activity. In another embodiment, the method includes decreasing PDGF activity. In still another embodiment, the method includes decreasing IL-1 activity. In some embodiments, the method includes decreasing expression of TNF, PDGF and/or IL-1. In one embodiment, the method includes decreasing expression of TNF. In another embodiment, the method includes decreasing of PDGF activity. In yet another embodiment, the method includes decreasing expression of IL-1.

The disclosure also provides a method of decreasing invasiveness or migration of a fibroblast-like synoviocyte, the method including contacting fibroblast-like synoviocytes with an effective amount of an RPTPα antagonist. Invasiveness or migration of FLS can be measured in patients by sampling their FLS. In some embodiments, a subject or patient could be selected for treatment based on their FLS characteristics, e.g. invasiveness or migration. FLS may also be assessed using PET-imaging and FLS-targeted tracers.

In various embodiments of the foregoing, the fibroblast-like synoviocyte (FLS) is a rheumatoid arthritis fibroblast-like synoviocyte. The term “rheumatoid arthritis fibroblast-like synoviocyte” refers to an FLS constituted within or obtained from a subject having rheumatoid arthritis or an FLS that causes, extends or exacerbates RA or symptoms thereof. In some embodiments, the fibroblast-like synoviocyte expresses high levels of RPTPα relative to a standard control (e.g., a non-rheumatoid arthritis fibroblast-like synoviocyte).

Further to any aspect or embodiment disclosed above, the RPTPα antagonist can be an anti-RPTPα antibody, an extracellular domain of RPTPα, or an RPTPα ligand mimetic.

In one embodiment, the anti-RPTPα antibody is an anti-RPTPα extracellular antibody. The term “extracellular antibody” in this context refers to an antibody which is directed to an extracellular portion of a target molecule. RPTPα is expressed as a transmembrane precursor protein that undergoes proteolytic cleavage to generate two non-covalently attached subunits, an N-terminal extracellular subunit, and a C-terminal subunit containing the intracellular and transmembrane regions and a small extracellular region. Thus, an anti-RPTPα extracellular antibody is directed to the extracellular portion of RPTPα. In one embodiments, the anti-RPTPα extracellular antibody is directed against (e.g., raised against or binds to) SEQ ID NO: 10, SEQ ID NO: 11, or SEQ ID NO: 12.

In other embodiments, the anti-RPTPα antibody is an anti-RPTPα dimer inhibiting antibody or an anti-RPTPα declustering antibody. The term “dimer inhibiting antibody” refers, in the usual and customary sense, to an antibody which binds a target thereby inhibiting dimerization of the target to its cognate thus preventing dimer formation. The term “declustering antibody” refers, in the usual and customary sense, to an antibody (e.g., including a multivalent antibody, e.g., a divalent antibody) which can prevent or reduce RPTPα clustering. In yet other embodiments, the anti-RPTPα antibody is an anti-RPTPα dimer-inhibiting antibody. In still other embodiments, the anti-RPTPα antibody is an anti-RPTPα declustering antibody. In one embodiment, the disclosure relates to an antibody or a functional equivalent thereof that specifically recognizes and binds to at least part of an epitope recognized by the monoclonal antibody 2F8. In one embodiment, the anti-RPTPα antibody binds to an epitope of RPTP located within the amino acid sequence SEQ ID NO: 1 or SEQ ID NO: 2. In other embodiments, the anti-RPTPα antibody binds to an epitope of RPTPα located within the amino acid sequence of SEQ ID NO: 3, SEQ ID NO: 4, SEQ ID NO: 5, SEQ ID NO: 6, SEQ ID NO: 7, SEQ ID NO: 8, or SEQ ID NO: 9.

The disclosure provides isolated peptides comprising a peptide sequence of at least 5 amino acids present in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, or SEQ ID NO: 9, said peptides being useful for raising antibodies to the peptides. In one embodiment, the disclosure provides antigenic peptides of at least 5 amino acids present in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, or SEQ ID NO: 9. By “present in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, or SEQ ID NO: 9” is meant that it is a peptide between 5 and 35 amino acids in length. The peptide can be 5-10, 5-15, 5-20, 5-25, 5-30, 5-35, 10-15, 10-20, 10-25, 10-30, 10-35, 15-20, 15-25, 15-30, 15-35, 20-25, 20-30, 20-35, 25-30, or 30-35 amino acids in length.

The disclosure provides an antibody or functional equivalent or fragment thereof that specifically binds to an epitope, which has an amino acid sequence present in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, or SEQ ID NO: 9 or an amino acid sequence comprising the amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, or SEQ ID NO: 9. Such an antibody may be a polyclonal antibody, or a monoclonal antibody, or a fragment of either of the foregoing so long as it retains the characteristics to bind to the epitope, which has an amino acid sequence present in SEQ ID NO:1, SEQ ID NO: 2, SEQ ID NO: 3, SEQ ID NO: 4, SEQ ID NO: 5, SEQ ID NO: 6, SEQ ID NO: 7, SEQ ID NO: 8, or SEQ ID NO: 9 or an amino acid sequence comprising the amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, or SEQ ID NO: 9. In still other embodiments, the antibody or its fragment includes, without limitation, single-chain antibodies, diabodies, triabodies, tetrabodies, Fab fragments, F(ab′) 2 fragments, Fd, scFv, domain antibodies, bispecific antibodies, minibodies, scAb, IgD antibodies, IgE antibodies, IgM antibodies, IgG1 antibodies, IgG2 antibodies, IgG3 antibodies, IgG4 antibodies, derivatives of constant regions of the antibodies, and artificial antibodies based on protein scaffolds, so long as they have a binding activity to the epitope, which has an amino acid sequence present in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, or SEQ ID NO: 9 or an amino acid sequence comprising the amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, or SEQ ID NO: 9. Antibodies having mutations in variable regions thereof are encompassed within the scope of the present disclosure so long as they retain their characteristics of binding to the epitope described herein. As an example, such antibodies may include conservatively modified variants. Such modifications in the antibody will not cause any change in its characteristics. As a result, a method of producing an antibody or conservatively modified variants thereof falls within the scope of this disclosure.

An antibody binding to one or more epitopes of this disclosure, which has or have an amino acid sequence present in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, or SEQ ID NO: 9 or an amino acid sequence comprising the amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, or SEQ ID NO: 9 may be obtained using methods known in the art. The antibody may be prepared by inoculating an animal with said epitope, a complex including said epitope, or a polynucleotide encoding the epitope and producing and panning for an antibody specifically binding to the epitope from the inoculated animal.

An antibody according to the disclosure binds to RPTPα. Antibodies can show species-specificity for RPTPα. In the disclosure, high specificity for human RPTPα is useful. However, depending on the degree of sequence identity between RPTPα homologs of different species, a given antibody or epitope-binding fragment may show cross-reactivity with RPTPα from one or more other species. In some embodiments, it may be desirable to have such cross-reactivity, e.g., when testing antibodies in animal models of a particular disease or for conducting toxicology, safety and dosage studies.

In other embodiments, the antibody is a monoclonal antibody, a chimeric antibody, a recombinant antibody and/or a human or humanized antibody. A monoclonal antibody (mAb) is a single molecular species of antibody and is usually produced by creating hybrid antibody-forming cells from a fusion of non-secreting myeloma cells with immune spleen cells. Polyclonal antibodies, by contrast, are produced by injecting an animal (such as a rodent, rabbit or goat) with an antigen, and extracting serum from the animal. A chimeric antibody is an antibody in which the variable domain of e.g. a murine antibody is combined with the constant region of a human antibody. Recombinant antibodies are obtained via genetic engineering without having to inject animals. Human antibodies according to the disclosure may be prepared using transgenic mice or by phage display; these methods are well known in the art.

In some embodiments, the RPTPα antagonist is an RPTPα ligand mimetic, wherein the anti-RPTPα ligand mimetic is a peptide or a small chemical molecule. In other embodiments, the RPTPα antagonist is the extracellular domain of RPTPα. In certain embodiments, the RPTPα antagonist comprises a peptide with the sequence of SEQ ID NO: 10, SEQ ID NO: 11, or SEQ ID NO: 12.

In another embodiment, there is provided a pharmaceutical composition including an RPTPα antagonist and a pharmaceutically acceptable excipient. In some embodiments, the pharmaceutical composition comprises an antibody that binds to one or more epitopes and/or antigenic peptides of this disclosure, which has or have an amino acid sequence present in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, or SEQ ID NO: 9 or a peptide comprising the amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, or SEQ ID NO: 9 and a pharmaceutically acceptable carrier. In still other embodiments, the pharmaceutical composition comprising an antibody can comprise one or more polyclonal or monoclonal antibodies which bind to one or more epitopes or antigenic peptides of this disclosure. Formulations of antibodies of this disclosure may be prepared by mixing with acceptable carriers, excipients, or stabilizers in the form of, e.g., lyophilized powders, slurries, aqueous solutions or suspensions. In one embodiment, antibodies of this disclosure are diluted to an appropriate concentration in a histidine buffer with NaCl or sucrose (e.g., 2-15% (w/v)) optionally added for tonicity. Additional agents, such as polysorbate 20 or polysorbate 80, at 0.01 to 0.10% (w/v) may be added to enhance stability.

In some embodiments, the pharmaceutical composition is for treating an individual who has a disease by administering to the individual a pharmaceutical composition including a therapeutically effective amount of an RPTPα antagonist and a pharmaceutically acceptable excipient. In other embodiments, the pharmaceutical composition is for treating an individual who may be at risk of developing a disease by administering to the individual a pharmaceutical composition including a therapeutically effective amount of an RPTPα antagonist and a pharmaceutically acceptable excipient. In some embodiments, the disease is an autoimmune disease or disorder, an inflammatory disease or disorder, a fibrotic disease or cancer (e.g., neoplasm, solid tumor, or cell proliferative disorder). In other embodiments, the disease is an inflammatory autoimmune disease (IAD). In still other embodiments, the disease is a disease associated with a patient's or subject's joints. In a certain embodiment, the inflammatory autoimmune disease is rheumatoid arthritis.

In yet other embodiments, the pharmaceutical composition is useful for treating an individual who has or may be at risk of developing a fibrotic disease or disorder. In a further embodiment, the disease is an autoimmune fibrotic disease or disorder. In yet a further embodiment, the disease in a non-autoimmune fibrotic disease or disorder. In a still further embodiment, the fibrotic disease is associated with a patient's skin or dermis. In yet another embodiment, the fibrotic disease is associated with a patient's lungs. In still other embodiments, the fibrotic disease is associated with a patient's internal organs, for example, kidney. In a certain embodiment, the fibrotic disease is systemic sclerosis. In a certain embodiment, the fibrotic disease is pulmonary arterial fibrosis.

In still other embodiments, the pharmaceutical composition is useful for treating an individual who has or may be at risk of developing an autoimmune disease, an inflammatory autoimmune disease, a fibrotic disease, or cancer. In one embodiment, the pharmaceutical compositions are useful for treating an individual who has an autoimmune disease, an inflammatory autoimmune disease, a fibrotic disease, or cancer by administering to the individual a pharmaceutical composition including a therapeutically effective amount of an RPTPα antagonist and a pharmaceutically acceptable excipient. In one embodiment, the pharmaceutical compositions are for treating an individual who may be at risk of developing an autoimmune disease, an inflammatory autoimmune disease, a fibrotic disease, or cancer by administering to the individual a pharmaceutical composition including a therapeutically effective amount of an RPTPα antagonist and a pharmaceutically acceptable excipient. In yet another embodiment, the inflammatory autoimmune disease is an arthritis. In still another embodiment, the autoimmune disease is fibroblast mediated disease, arthritis, osteoarthritis, rheumatoid arthritis, psoriatic arthritis, juvenile idiopathic arthritis, multiple sclerosis, systemic lupus erythematosus (SLE), myasthenia gravis, juvenile onset diabetes, diabetes mellitus type 1, Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, psoriasis, ichthyosis, Graves ophthalmopathy, inflammatory bowel disease, Addison's disease, Vitiligo, asthma, scleroderma, systemic sclerosis, or allergic asthma. In one embodiment, the autoimmune disease is rheumatoid arthritis. In another embodiment, the fibrotic disease is a non-autoimmune disease. In still another embodiment, the fibrotic disease is associated with a patient's skin. In yet another embodiment, the fibrotic disease is associated with a patient's lungs. In still another embodiment, the fibrotic disease is associated with a patient's internal organs, for example, kidney. In a certain embodiment, the fibrotic disease is systemic sclerosis. In a certain embodiment, the fibrotic disease is pulmonary arterial fibrosis. In one embodiment, the cancer is lung (small cell and non-small cell) cancer, thyroid cancer, prostate cancer, pancreatic cancer, breast cancer, ovarian cancer, colon cancer, sarcoma or melanoma. In still another embodiment, the cancer is of blood cancer, brain cancer, leukemia, chronic lymphocytic leukemia, chronic myeloid leukemia, colorectal cancer, gastrointestinal stromal tumor, kidney cancer, lymphoma, or multiple myeloma.

The compositions disclosed herein can be administered by any means known in the art. For example, compositions may include administration to a subject intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intrathecally, subcutaneously, subconjunctival, intravesicularlly, mucosally, intrapericardially, intraumbilically, intraocularly, orally, locally, by inhalation, by injection, by infusion, by continuous infusion, by localized perfusion, via a catheter, via a lavage, in a creme, or in a lipid composition. Administration can be local, e.g., to the joint, skin or systemic.

Solutions of the active compounds as free base or pharmacologically acceptable salt can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations can contain a preservative to prevent the growth of microorganisms.

Pharmaceutical compositions can be delivered via intranasal or inhalable solutions or sprays, aerosols or inhalants. Nasal solutions can be aqueous solutions designed to be administered to the nasal passages in drops or sprays. Nasal solutions can be prepared so that they are similar in many respects to nasal secretions. Thus, the aqueous nasal solutions usually are isotonic and slightly buffered to maintain a pH of 5.5 to 6.5. In addition, antimicrobial preservatives, similar to those used in ophthalmic preparations, and appropriate drug stabilizers, if required, may be included in the formulation. Various commercial nasal preparations are known and can include, for example, antibiotics and antihistamines.

Oral formulations can include excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders. In some embodiments, oral pharmaceutical compositions will comprise an inert diluent or assimilable edible carrier, or they may be enclosed in hard- or soft-shell gelatin capsule, or they may be compressed into tablets, or they may be incorporated directly with the food of the diet. For oral therapeutic administration, the active compounds may be incorporated with excipients and used in the form of ingestible tablets, buccal tablets, troches, capsules, elixirs, suspensions, syrups, wafers, and the like. The percentage of the compositions and preparations may, of course, be varied and may conveniently be between about 2 to about 75% of the weight of the unit, or between 25-608. The amount of active compounds in such compositions is such that a suitable dosage can be obtained.

For parenteral administration in an aqueous solution, for example, the solution should be suitably buffered and the liquid diluent first rendered isotonic with sufficient saline or glucose. Aqueous solutions, in particular, sterile aqueous media, are especially suitable for intravenous, intramuscular, subcutaneous and intraperitoneal administration. For example, one dosage could be dissolved in 1 ml of isotonic NaCl solution and either added to 1000 ml of hypodermoclysis fluid or injected at the proposed site of infusion.

Sterile injectable solutions can be prepared by incorporating the active compounds or constructs in the required amount in the appropriate solvent followed by filtered sterilization. Generally, dispersions are prepared by incorporating the various sterilized active ingredients into a sterile vehicle which contains the basic dispersion medium. Vacuum-drying and freeze-drying techniques, which yield a powder of the active ingredient plus any additional desired ingredients, can be used to prepare sterile powders for reconstitution of sterile injectable solutions. The preparation of more, or highly, concentrated solutions for direct injection is also contemplated. DMSO can be used as solvent for extremely rapid penetration, delivering high concentrations of the active agents to a small area.

In some embodiments, the pharmaceutical compositions are formulated as a spray, cream, an emulsion, a microemulsion, a gel (e.g., a hydrogel, an organogel, an inorganic or silica gel, a high-viscosity gel or a low-viscosity gel), a lotion, a lacquer, an ointment, a solution (e.g., a moderate to highly viscous solution), or a transdermal patch. In a suitable embodiment, the composition is a gel, for example, a low-viscosity gel or a spray. Alternatively, the composition is a high-viscosity gel. The pharmaceutical composition of the dislcloaure may also be formulated as a transdermal patch. Low viscosity gels are, for example, gels having a dynamic viscosity in the range of about 400-4000 cP at STP. High viscosity gels are, for example, gels having a dynamic viscosity of at least 4000 cP at STP. Methods of preparing compositions for topical administration are known in the art (see, for example, Remington's Pharmaceutical Sciences, 2000-20th edition, and The United States Pharmacopeia: The National Formulary, USP 24 NF19, published in 1999).

There are provided methods of treating, preventing, and/or ameliorating an autoimmune disorder in a subject in need thereof, optionally based on the diagnostic and predictive methods described herein. The course of treatment is best determined on an individual basis depending on the particular characteristics of the subject and the type of treatment selected. The treatment, such as those disclosed herein, can be administered to the subject on a daily, twice daily, bi-weekly, monthly or any applicable basis that is therapeutically effective. The treatment can be administered alone or in combination with any other treatment disclosed herein or known in the art. The additional treatment can be administered simultaneously with the first treatment, at a different time, or on an entirely different therapeutic schedule (e.g., the first treatment can be daily, while the additional treatment is weekly).

Administration of a composition for ameliorating an autoimmune disease, an inflammatory autoimmune disease, a fibrotic disease, or cancer can be a systemic or localized administration. For example, treating a subject having an autoimmune disease, an inflammatory autoimmune disease, a fibrotic disease, or cancer can include administering an oral or injectable form of an RPTPα antagonist on a daily or weekly schedule.

Any appropriate element disclosed in one aspect or embodiment of a method or composition disclosed herein is equally applicable to any other aspect or embodiment of a method or composition. For example, the therapeutic agents set forth in the description of the pharmaceutical compositions provided herein are equally applicable to the methods of treatment and vice versa.

The invention is illustrated in the following examples, which are provided by way of illustration and are not intended to be limiting.

### EXAMPLES

Mouse work and mouse fibroblast like synoviocyte (FLS) culture.

RPTPA knockout (KO) mice on C57BL/6 background were obtained from Dr. Ari Elson (Weizmann Institute) and bred to obtain littermate wild-type (WT) or RPTPA KO mice.

FLS lines were isolated from knee and ankle joints of 8-week old WT or RPTPA KO mice (as described in Svensson, M N D, et al. 2020 Sci Adv. 2020 Jun. 26: eaba4353). FLS were cultured in Dulbecco's modified Eagle's medium (DMEM; Fisher Scientific 10-017) with 10% fetal bovine serum (FBS; Omega Scientific), 2 mM L-glutamine, 100 units/ml penicillin, 100 g/ml streptomycin and 50 microg/ml Gentamicin (Life Technologies) at 37° C. in a humidified 5% CO2 atmosphere. For all experiments, FLS were used between passages 3 and 10, and subjected to overnight starvation in 0.1% FBS (serum-starvation medium) before functional assays.

### FLS Transfection and Transduction

FLS transfection was performed using Lipofectamine 3000 (Invitrogen L3000015) in DMEM with no additional additives once monolayer reached approximately 90% confluency. The transfection media was replaced with normal FLS media 3 hours after transfection. For assays, 36 or 48 hours after transfection, FLS were starved for 12 hours in serum-starvation medium.

### Antibodies.

The mouse anti-FLAG antibody (F3165) was from Sigma Aldrich, rabbit anti-SRC (2108S) and mouse anti-HA Alexa 488 ((2350S) were from Cell Signaling Technology (CST), goat anti-mouse Alexa 568 (A11031) and goat anti-rabbit Alexa 488 (A11008) were from Invitrogen. The hamster anti-mouse anti-RPTPα hybridoma #2F8 has been described (Su, J, et al. 1999 Curr Biol. 9 (10): 505-11). The hybridoma was cultured in Iscove's Modified Dulbecco's Medium (IMDM; Gibco 12440-053) with 10% fetal bovine serum (FBS; Omega Scientific), 2 mM L-glutamine, 2 mM Sodium Pyruvate, 100 units/ml penicillin, 100 g/ml streptomycin and 50 μg/ml Gentamicin (Life Technologies) at 37° C. in a humidified 5% CO2 atmosphere. Cells were collected into 50 ml tubes and centrifuged, then resuspended with IMDM with 1% FBS and cultured for 7 days. The supernatant was collected and the anti-RPTPα antibody was isolated from the supernatant with Protein G Sepharose.

### Plasmids.

WT-RPTPA (NM 008980.2) was cloned into a pcDNA3.1 (+) backbone between the AflII and XhoI restriction sites in frame with either C-terminal FLAG or HA tags.

### Transwell Migration Assays

Confluent FLS were harvested by light trypsin digestion and seeded at 2.5×10⁴ cells in 100 μl serum-free DMEM containing 0.5% bovine serum albumin (BSA) in the upper chamber of a 6.5 mm-diameter Transwell polycarbonate culture insert (Costar) with a pore size of 8 microns. Inserts were placed in 24-well plates with 600 μl DMEM containing 5% FBS. The assay plates were incubated for 4 h, after which the Transwell inserts were removed and the upper chamber gently wiped with a cotton swab to remove non-migrating cells. Transwell membranes were fixed for 10 min in prechilled (−80° C.) methanol and stained for 10 min in 0.5% crystal violet in 25% methanol. Cells were then rinsed and imaged using a light microscope at 10×. Cell migration was quantified by counting 4 random fields.

### Wound Healing Assay.

RPTPA KO FLS were plated into 6 well plates and allowed to grow to 90% confluency before transfection as described above. 36 h post transfection, cells were starved for 12 h, and a scratch wound was induced by drawing a micropipette tip through the middle of the coverslip. Pictures of the wound were acquired and the distance between the scratch margins marked to constitute the 0 h timepoint. The wound was allowed to heal for 24 h in high serum (20% FBS) media before acquiring another set of pictures of the same wound for the 24 h timepoint. Slides were then fixed and stained with crystal violet as described above and cells were visualized using a light microscope at 4× magnification. Each scratch wound was scored at four separate locations by ImageJ distance quantification.

### Western Blotting

Cells were lysed with a high SDS denaturing buffer (1×Laemmli sample buffer, 5% betaME, 2.05% SDS) and the gel was transferred at 40 volts at 60° C. for 1.5 h. Primary antibodies were diluted 1:1000 and secondary antibodies were diluted 1:3000 or 1:5000.

### Nikon N-STORM and TIRF Imaging and Imaris Analysis.

RPTPA KO mouse FLS were directly plated onto coverslips and allowed to grow to confluence in a 24 well plate. Cells were transfected once they had reached 90% confluency and starved for 12 h to 36 h post transfection as described above. After starvation, a scratch wound was induced as described above. The wound was allowed to close for 12 h in high serum media (20% FBS) before cell fixation with 4% paraformaldehyde. The cells were then permeabilized by 0.2% triton-X and stained with AF647 conjugate anti-FLAG Ab, AF488 conjugate anti-SRC Ab and phalloidin FITC reagent (for F-actin). The coverslips were then mounted onto slides with molecular probe gold reagent. Cells were imaged using 100×(1.49 NA) Apo TIRE objective with TIRF illumination on a Nikon Ti super-resolution microscope. Images were collected on an ANDOR IXON3 Ultra DU897 EMCCD camera using the multicolor sequential mode setting in the NIS-Elements AR software (Nikon Instruments Inc., NY). The power on the 488, 561 and 647 nm lasers was adjusted to 50% to enable collection of between 100 and 300 molecules per 256×256 camera pixel frame in the center of the field at appropriate threshold settings for each channel. Collection was set to 20,000 frames, yielding 1-2 million molecules, and the super-resolution images were reconstructed with the Nikon STORM software.

The position of individual molecules was localized with high accuracy by switching them on and off sequentially using the 488, 561 and 647 nm lasers at appropriate power settings. The position determined from multiple switching cycles can show a substantial drift over the duration of the acquisition. This error was reduced by correcting for sample drift over the course of the experiment by an auto-correlation method based on by correlating STORM images reconstructed from 200-1000 frames to that from the beginning of the acquisition. The number of frames used in a set was based on the number of molecules identified, and by default it was set to 10000 molecules. Displacement was corrected by translational displacement in the X, Y direction for 2D STORM. Axial drift over the course of the acquisition was minimized by engaging the Nikon perfect focus system. Calibration of chromatic shift (warp correction) was carried out using multicolored 100 nm TetraSpeck beads using minimum density per field of over 100 beads and using the 2D warp calibration feature of the Nikon STORM software.

Briefly, a total of 201 images were collected for each of the color channels (488, 561 and 647 nm) paired with NBD Phallicidin for F-actin, [SRC or PTP]-Alexa 568 and PTP-Alexa 647 respectively, without the cylindrical lens in place. Frames 1-20 and frames 182-201 were collected at the focal position. Frames 21-181 were collected across a range of 1.6 microns in 10 nm steps in the Z (covering 800 nm above and 800 nm below the focal plane). The calibration files generated from this macro (software feature) were applied during analysis for the correction of the STORM images. All fixed and stained samples were incubated in the following blinking solution (STORM buffer: 50 mM Tris, pH 8.0, 10 mM NaCl, 10% glucose, 0.1 M mercaptoethanolamine (MEA), 56 units/ml glucose oxidase, and 340 units/ml catalase. The STORM buffer was prepared by adding 100 microliter MEA solution and 10 microliter GLOX sample (14 mg glucose oxidase [from Aspergillus niger-type VII, Sigma-Aldrich Cat #G2133] and 1 mg catalase [from bovine liver, Sigma-Aldrich Cat #C40] dissolved in 250 microliters of 10 mM Tris, 50 mM NaCl pH 8.0) to 890 microliter of buffer B (50 mM Tris, 10 mM NaCl, 10% Glucose, pH 8.0) just before imaging.

Blinking events were followed for successive frames to ensure single molecule isolation by filtering out molecules with traces longer than 5 frames during analysis. Moreover, individual molecules were localized using point spread function (PSF) width filters of 200-400 nm based on a 100×(1.49 NA) objective and restrictions were placed on photon count signals associated with camera noise of the ANDOR electron multiplying charge-coupled device (EM-CCD) camera (estimated at 100 intensity units above 0). The data was further filtered based on empirical observation of photon count signals (peak height when converted to an intensity value) found in cells vs background staining on the glass slide surface (generally values above 300-700 intensity units above camera noise). The precision of the localization during a switching cycle is calculated from these multiple parameters and from photon counts using molecules that are ultimately well separated in the sample itself.

TIRF microscopy experiments were performed on the same multi-adapted N-STORM system using a 100×(1.45 NA) TIRF objective (Nikon Instruments, Melville, NY) on a Nikon TE2000U microscope custom modified with a TIRF illumination module as described. Images were acquired on a 14-bit, cooled CCD camera (Hamamatsu) controlled through NIS-Elements software. After placing the cells on the stage, the position of the individual laser beams was adjusted with the TIRF illuminator to impinge on the coverslip at an angle to yield a calculated evanescent field depth of a 70-100 nm for TIRF microscopy modes, staying as close to the lamellipodia to glass surface as possible and maintained for subsequent N-STORM data collection. Cells only at the wound edge with a distinct and characteristic classical migrating phenotype with a ruffling and protruding lamellipod in the direction of the wound were captured for NBD Phallicidin (Green), RPTPα (Far Red) and SRC (Red), prior to N-STORM imaging to be used in image processing as references of large biological compartments, like basal stress fibers, ruffles and leading edges.

Images obtained on the N-STORM system were exported as pointillism or localization coordinate map text files, which represent positions of individual blinks that have been localized with high accuracy by switching them on and off using the 488, 561 and 647 nm lasers. The localization coordinates were imported into the Imaris software (Bitplane, Inc.) where each blink was reconstructed as a sphere (spot) on an image grid, its centroid is the central coordinate position in three-dimensional space, and the diameter of the sphere (spot) is the localization accuracy error. The widefield TIRF images were also imported into Imaris as separate channels, aligned and fitted to overlay with the N-STORM image. This is used to define cellular polarity, compartments and structures using the lesser resolved original fluorescent signals. Once imported into Imaris all spots were filtered for a minimum localization accuracy (diameter 0-100 nm) and whether they, between the same population (RPTPα or SRC) are clustered in pairs of 3, 6 or 9 molecules to filter single or lone stray molecules. However, all localizations are considered in calculations to ensure consistency between experiments. Furthermore, all spots created were analyzed using the colocalized spots module in Imaris, to mark and score paired spots between separately labelled populations of RPTPα to RPTPα or RPTPα to SRC that lie within a defined distance from each other in three-dimensional space based on their centroid.

### Forster Resonance Energy Transfer (FRET) Methods.

Cells were plated, transfected and fixed as described above, followed by staining with primary, secondary, and direct conjugate antibodies.

Sensitized Emission (SE) Confocal FRET cFRET) and Precise FRET (pFRET).

All images were acquired with a Zeiss laser scanning confocal microscope (LSM) 880 Airyscan using a 63×(1.46 NA) and a 63×(1.4 NA) objective using the 32-channel GaAsP-PMT area detector. In the Zen software, the capture method was designed to sequentially capture the donor channel [488 excitation, and emission 508-535 (27 lambda)], raw FRET (R-FRET) channel [488 excitation and emission 588-624] and acceptor channel [568 excitation and emission 588-624)]. All 12-bit images were acquired with Nyquist resolution parameters using optimal pinhole sizes (pixel size 0.060×0.060 microns) and optimal frame size of 2644×2644. All 12-bit images were acquired using the full dynamic intensity range (0-4096) that was determined with the population of WT cells having the moderate to brightest signal expression of PTP and or SRC. For cFRET and pFRET imaging, the system consisted of a 45-mW argon laser (458, 488, 514 nm), a 10-mW diode solid state laser (561 nm) and a He-Ne 633-nm laser. System settings were set as constants and defined based on the WT constructs: 1% laser power for acceptor, and 3% for both R-FRET channel and donor channels, detector signal amplification (digital gain) was set to 900V for all acquisitions, donor, raw FRET and acceptor. Both donor-acceptor samples and donor alone as well as acceptor alone controls were captured for every experiment in order to later incorporate background subtraction and spectral bleed through correction factors into the R-FRET samples using established automated algorithm methods of analysis. Algorithms to remove spectral bleedthrough/cross-talk and correct for spectral variations in donor and acceptor channels, autofluorescence, background fluorescence, detector and optical noise are all established components of cFRET and pFRET methodologies. Using imageJ, a creation of automated and scripted of the cFRET method was developed earlier in both widefield and confocal platforms. In addition, all images were also processed in order to calculate FRET efficiencies of all cells at the leading edge using the method of pFRET that was developed and scripted by Amassi Periasamy's group and incorporated into a complete data processing software package of imageJ plugins written in JAVA.

Final processed cFRET Images were further analyzed in Image Pro Premier 10 to define area and polarity of FRET positive regions of interest (ROIs) at the leading edge cell periphery. In order to automate and define distinct and broad FRET positive zone groupings for this assay, as was previously done, images were converted to 8-bit greyscale and the FRET positive zones were auto-outlined using Image Pro Premier software tools (wand). Using this technique, the dynamic range of significant FRET positive signal (compared between FRET methods) was between 85-256 for all samples and was used to outline FRET positive regions of interest (ROIs) that were scored for each quadrant and charted in excel. The same original raw data was also further processed in the pFRET software and % FRET efficiencies were obtained for each cFRET sample post-processed for FRET positive zone area analysis.

### Spectral Processed Acceptor Photobleaching (AP) FRET.

All images were acquired with a Zeiss LSM 880 Airyscan using a 63×(1.46 NA)/or a 63×(1.4 NA) objective and the 32-channel GaAsP-PMT area detector. Using the spectral detector, as outlined in, donor alone and acceptor alone samples are acquired using the 32 channel spectral detector to establish emission curves that are then incorporated into the inherent on-line fingerprinting mode to auto correct for spectral bleedthrough The LSM 880 is designed with acousto-optic tunable filters that can be used to obtain images at a series of discrete 10 nm wavelength bands, generating lambda stacks. The spectral signatures for the individual 488 and 568 fluorophores and background signals are obtained from these lambda stacks and linearly unmixing is employed within the Zen software, to segregate mixed (overlapping) fluorescent signals to obtain the spatial contribution for each fluorophore known as emission online fingerprinting.

FRET efficiency using apFRET was calculated by measuring the difference between the quenched donor signal in the presence of the acceptor, and the dequenched donor signal after the acceptor has been bleached. FRET efficiency is defined over a time regimen of 30 cycles (roughly 300s) post bleaching, after 4 unbleached scans are acquired to establish a stable baseline stable fluorescence at each ROI selected. The Software module in Zen automatically tracks the mean changes in fluorescence intensity within the ROIs and is thus used to calculate the % FRET efficiency as outlined previously by others.

Briefly, this is mathematically based on the % of donor fluorescence recovered between t=0 (the average of 4 unbleached scans) and the plateau peak of donor fluorescent recovery at approximately t=15m later. Regions far away from the leading edge that may dilute the overall FRET efficiency at the leading edge are omitted.

### Fluorescence Lifetime Imaging Microscopy FRET (FLIM-FRET).

All the time-domain FLIM images were acquired with a Leica time-correlated single photon counting (TCSPC) SP8X Fast Lifetime Contrast (FALCON) system from Leica Microsystems (Mannheim, Germany). This integrated fluorescent lifetime (TCSPC-FLIM) system utilizes a confocal scan head with field-programmable gated array (FPGA) electronics, pulsed laser excitation and fast, spectral single-photon counting detectors. The signals of both laser pulses and photon arrival pulses from each detector are digitized at very high speed with a temporal resolution of 97 ps. These direct measurements from the differences in arrival times between detection and excitation pulses are rendered directly online as ‘fast FLIM’ images, in a time resolved manner.

Donor alone was imaged from FLS labeled with anti-HA (A488) (Control) while donor and acceptor were imaged from FLS labeled with anti-HA (A488) and anti-FLAG (A568) (FRET pair). Prolonged gold mounted samples were imaged under a 100×(1.4 NA) oil immersion objective. Donor excitation was achieved using a white light pulsed laser (laser power at 50%, 1.5-2 mW per line) tuned at 40 MHz coupled with single photon counting electronics and subsequently detected by highly sensitive hybrid internal detectors in photon counting mode. Collection parameters included: acquiring 8-bit (all converted internally to 16-bit final image) images pixel by pixel (512× 512 frame size) with an emission collection detection range of 498-584 nm, a detector gain set at 100 and scan speed at 400 Hz. Frame acquisitions averaging was 100 to accumulate enough photons at the cell periphery and at lamellipodia ruffling edges. As described in detail in, to remove artefacts caused by noise or photo-bleaching and insufficient signal to noise, cells with negligible amounts of bleaching and at least 200-1000 photons per pixel were only allowed in the analysis. The acquired fluorescence decay of each pixel was deconvoluted with the instrument response function (IRF) and then post processed by being fitted with two-exponential theoretical models using Leica Application Suite X (LAS X) from Leica Microsystems. Using the Leica Applications suite, 2D phasor plot analysis loops were used to segregate single donor populations from FLIM-FRET populations.

### Statistical Analysis.

All statistical analyses were performed using GraphPad Prism 5 software. Statistical comparisons between 2 groups were performed using Mann-Whitney U test except for comparisons of spectral FRET data where ordinary one-way ANOVA followed by Dunnett's multiple comparison test of the area under curves (AUC) was used. For multiple comparisons, the Kruskal-Wallis test was used. P values less than 0.05 were considered significant.

Super-Resolution Microscopy Studies of RPTPα Clustering and association with SRC in migrating FLS.

Stochastic optical reconstruction microscopy (STORM) was used to accurately spatially localize the target molecules in migrating FLS. FLS from Ptpra-KO mouse (αKO FLS) were transfected with plasmids expressing FLAG-tagged RPTPα-WT or the dimerization-impairing P210LP211L (P210) wedge mutant of RPTPα. After 12 hours starvation with 0.5% FBS media, FLS monolayers were scratch-wounded followed by stimulation of FLS migration with 20% FBS media for 12 hours. Cells were then fixed and stained with AF647 conjugate anti-FLAG antibodies, AF488 conjugate anti-SRC antibodies and phalloidin FITC reagent (for F-actin). Images of cells were captured using total internal resolution microscopy (TIRF) and localization microscopy through N-STORM and analyzed using the Imaris 9.7 software (Bitplane Inc). Cells at the wound edge with a classical migrating phenotype characterized by a ruffling and protruding lamellipod in the direction of the wound were captured in conventional TIRF microscopy for NBD Phallicidin (Green), RPTPα (Red) and SRC (orange), prior to N-STORM imaging. Widefield TIRF images were also imported into Imaris as separate channels and aligned to overlay with N-STORM images in order to define cellular polarity, compartments and structures using the lesser resolved original fluorescent signals in widefield. These included F-actin rich: basal stress (BSTRESS) fibers, aligned in the direction of migration, and leading edge (LEDGE) lamellipodia, ruffles and filopodia. From STORM data, a pointillism, or localization map was constructed in NIS Elements software (Nikon Instruments Inc., NY) and imported into Imaris software for further processing. Here each point represents the most likely location of the peak of a two-dimensional Gaussian distribution of photons generated from one fluorochrome: a blink. The imported localization coordinate map of RPTPα or SRC fluorescent N-STORM confirmed blinks, which were previously filtered for drift and background signals in the NIS Elements software are represented in Imaris as spheres whose diameter represents the localization accuracy and whose centroid is used to compare distances between same or different paired molecules.

In Imaris, the Colocalized Spots module was used to score the number of each pair of all RPTPα or SRC spots binned at defined nm distance intervals. 65 nm was adopted as the cut off distance separation to infer functionally relevant colocalization or closest realistic localization proximity (which will be referred to as colocalization from here on) between RPTPα and other RPTPα molecules. This distance was inferred from molecular modeling of RPTPα dimers bound to their respective fluorophore tags (AF488 and AF568) based on multiple crystal structures of RPTPα intracellular domain. The model assumed that helices al′ and x2′ from at least one of the D2 domains detach from the core PTP domain in order to allow dimerization of D1 and suggested a maximum theoretical distance between fluorophores of 55-70 nm, leading us to conclude that any distance >65 nm was not due to RPTPα dimerization. Modeling of the RPTPα-SRC association suggested a maximum theoretical distance between RPTPα and SRC fluorophores between 25 and 45 nm, so it was concluded that the >65 nm cutoff excluded functionally relevant colocalization between SRC and RPTPα as well as between SRC and other SRC molecules.

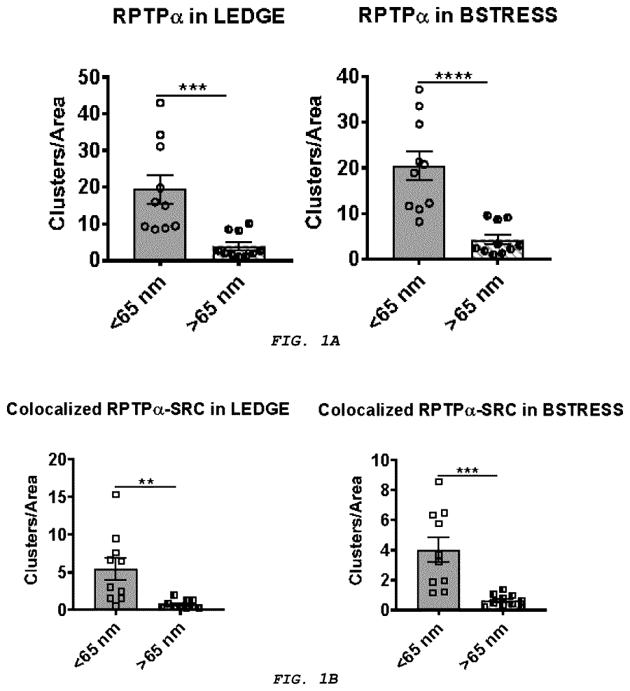

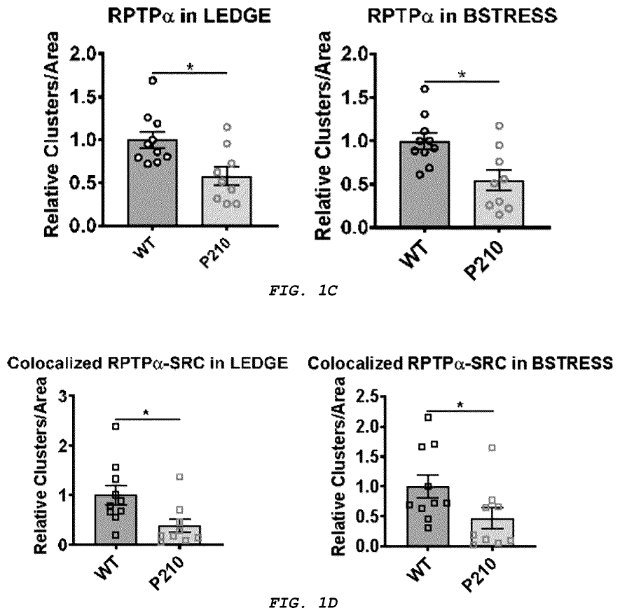

Total internal reflection microscopy (TIRF) micrograph (I) and Nikon system Stochastic Optical Reconstruction Microscopy (N-STORM) were used to produce images and localization maps of FLS migrating with leading edge (LEDGE) and polarized basal stress fibers (BSTRESS), using FLAG-tagged RPTPα along with HA-tagged SRC. LEDGE and BSTRESS are two distinct populations of actin-rich structures necessary for directed migration of FLS. The localization map, showing highly clustered RPTPα (distance <65 nm) (FIG. 1A) and co-localized RPTPα-SRC (distance <65 nm) (FIG. 1B), when overlaid on an Imaris-rendered iso-surfaced F-actin TIRF image, showed increased and relevant localizations within LEDGE and ruffles and considerably fewer outside these regions. The results of this super-resolution microscopy of RPTPα and SRC in migrating FLS showed that RTPRa dimer clusters interact with SRC along actin stress fibers.

FIG. 1C shows that colocalization of RPTPα was significantly reduced by the P210L/P211L mutation in the wedge region of RPTPα, which impairs RPTPα dimerization. FIG. 1D shows, unexpectedly, that this mutation also reduced the co-localization of RPTPα with SRC at BSTRESS and LEDGE stress fibers.

Sensitized Emission (SE) Confocal FRET (SECFRET) and Confocal Based Precise FRET (pFRET) Studies of RPTPα Clustering and Association with SRC in Migrating FLS.

FRET microscopy based analytical methods using antibodies (Ab) labeled with donor (AF488) or acceptor (AF568) fluorophores were employed. FRET occurs when the donor and acceptor fluorophores are very close to each other (within 10 nm) defining a dimerized or bound state for the molecules of interest. FRET images of cells at the leading edge of FLS migrating into a scratch-wound were processed through scripted macros in image. Bleed through and background corrections were made for each of the three acquired channel images, namely: image 1: donor excitation to donor emission; image 2: acceptor excitation to acceptor emission; image 3: donor excitation to acceptor emission. The final output-corrected FRET images were 8-bit and pseudo-colored to show signal hot spots of molecule clustering. These SE confocal FRET or SECFRET images were further analyzed in Image Pro Premier 10 to define the area of FRET positive regions of interest (ROIs). Briefly, each FLS was separated into 4 compartments: leading edge (towards the wound), trailing edge (away from the wound) and two lateral compartments. Then the total area of FRET positive zones were automatically outlined using smart segmentation modules in image pro and the results exported into excel.

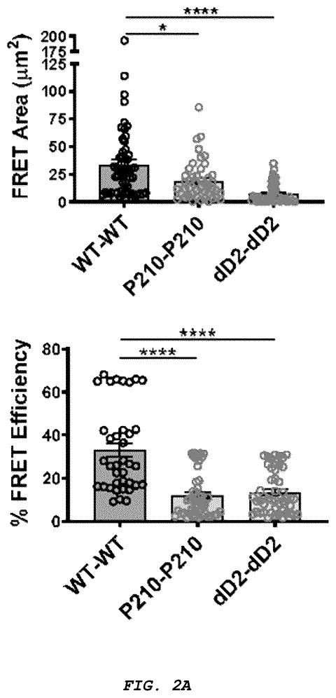

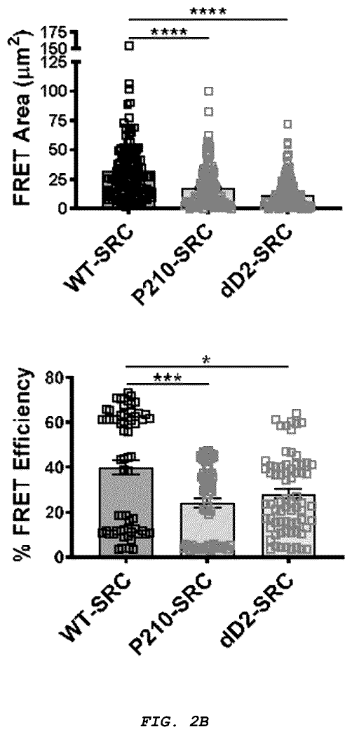

RPTPα-KO mouse (αKO) FLS were transfected with plasmids expressing one of the following: FLAG-tagged and HA-tagged RPTPα-WT, its P210LP211L (P210) mutant or a C-terminal truncation of RPTPα at aa 560 (dD2). Removal of the D2 domain has been reported to regulate dimerization of RPTPα via an oxidative mechanism and includes the putative SRC-recruiting Tyr825. For the assessment of RPTPα clustering, the oko cells were stained with AF488-labeled anti-HA Ab and indirectly AF568-labeled anti-FLAG Ab. For the assessment of RPTPα-SRC association, the same transfected αKO FLS were stained with AF488-labeled anti-SRC Ab and indirectly AF568-labeled anti-FLAG Ab. Experiments were conducted on 30-50 cells and four different αKO FLS lines per construct across 4-6 experimental repeats. Cells for FRET analysis were selected among the ones expressing relatively equal expression (intensity range 700-4095) of FLAG and HA based on their overall average acquisition mean fluorescence intensities. All acquisition settings, including laser power, detector gain and laser scan speed and averaging (pixel dwell time 1.26 ms), were the same for all variants and experiments. In FIG. 2A, HA-FLAG FRET occurred significantly at LEDGE of WT RPTPα-expressing cells (WT-WT) however the P210LP211L mutation (P210-P210 cells) and the dD2 truncation (dD2-dD2 cells) significantly reduced the area of FRET positive region at LEDGE as deduced using the SECFRET method. % SE FRET efficiency-calculated using the using the precise FRET (SEpFRET) method developed and scripted by Amassi Periasamy's group and incorporated into a complete data processing software package of imageJ plugins written in JAVA at LEDGE was also significantly reduced in P210-P210 and dD2-dD2 cells when compared to WT-WT cells demonstrating that the RPTP P210LP211L and dD2 mutations significantly reduce clustering of RPTPα in a primary cell model.

In FIG. 2B, the area of FRET signal and % FRET efficiency (via SEpFRET) of RPTPα association with SRC in LEDGE was substantially reduced in cells expressing the P210LP211L or the dD2 mutants of RPTPα (P210-SRC and dD2-SRC cells respectively) when compared to cells expressing WT RPTPα (WT-SRC cells), demonstrating that RPTPα clustering positively correlates with RPTPα and SRC co-localization.

Spectral and Fluorescence Lifetime Imaging Microscopy (FLIM) FRET Studies of RPTPα Clustering and Association with SRC in Migrating FLS.

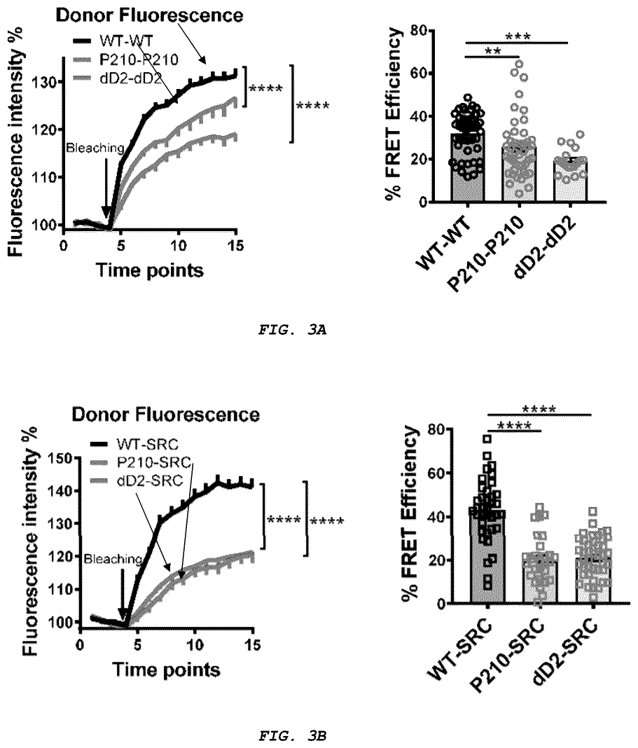

The applied spectral based acceptor photobleaching FRET (apFRET) and fluorescence lifetime imaging FRET (FLIM-FRET) were used to study RPTPα clustering and RPTPα-SRC association in migrating FLS. Spectral mediated FRET allows to separate the contributions of individual signals in each pixel and efficiently removes the contribution of donor spectral bleedthrough to the FRET signal yielding an at least partially corrected FRET image of the interacting pairs (RPTP-RPTPα or RPTPα-SRC) FRETing at the LEDGE of FLS migrating into the wound. Having collected prebleached images prior to photo bleaching followed by testing these for mathematically corrected FRET using the SECFRET and SEpFRET methodologies, there was high confidence that apFRET-selected cells had truly FRET positive leading edges. Next, FRET efficiency using apFRET was assessed by measuring the difference between the quenched donor signal in the presence of the acceptor, and the dequenched donor signal after the acceptor has been bleached. The advantage of this approach is that each cell serves as its own control. Using the photobleaching module in the Zen software, two ROIs were marked within the leading edge, one to bleach, another serving as an unbleached control, and also an area devoid of cells was selected serving as a background control. The software module in Zen automatically tracks the mean changes in fluorescence intensities within the ROIs which is used to calculate the % FRET efficiency. For accuracy, the subdivision of the LEDGE regions within the ROIs into smaller subdivisions allows for the stringent outline of the cell lamellipodium to include cellular and exclude non-cellular areas. Regions far away from the leading edge that may dilute the overall FRET efficiency at the leading edge were omitted from this analysis. Experiments were performed on FLS lines transfected with one of the following: empty vector, vector expressing WT RPTPα (WT-WT), vector expressing the P210LP211L mutant (P210-P210 cells) or vector expressing the dD2 truncation (dD2-dD2 cells), assessing 20-30 cells and four different αKO lines per construct across 4-6 experimental repeats. FIG. 3A shows significantly higher anti-HA donor fluorescence recovery after acceptor photobleaching and consequent % RPTPα-RPTPα FRET efficiency was observed at LEDGE of WT RPTPα-expressing cells (WT-WT) than in FLS expressing the P210LP211L mutant (P210-P210 cells) or the dD2 truncation (dD2-dD2 cells). Similarly, FIG. 3B shows that significantly higher anti-SRC donor fluorescence recovery after acceptor photobleaching and consequent % RPTPα-SRC FRET efficiency was observed at LEDGE of WT RPTPα-expressing cells (WT-SRC cells) than in FLS expressing the P210LP211L mutant (P210-SRC cells) or the dD2 truncation (dD2-SRC cells).

FLIM can be used to detect changes in donor lifetime that accompany energy transfer to the acceptor during FRET. However, the interpretation of fluorescence lifetime measurements is complicated by the fact that most of the fluorescent proteins that have been characterized in living cells exhibited multi-exponential fluorescence decays. To address these shortcoming the use of 2D graphical views of lifetime distributions in phasor plots inherent in the Leica applications software (Leica, Inc). In the phasor representation, the analysis of FLIM data is done by observing clustering of pixels values in specific regions of the phasor plot rather than by fitting the fluorescence decay using exponentials. The interpretation of the data is immediate since it does not require calculations or nonlinear fitting. FLIM data was collected from donor alone by staining FLS with anti-HA (A488) alone (unquenched donor,) and from donor and acceptor FRET pair by adding anti-FLAG A568. Phasor plot analysis loops were used to segregate single donor populations from FLIM-FRET populations. Donor alone samples or dequenched donor residing in photobleached areas in FRET pair WT-RPTPα samples yielded a no FRET population which was distinct from low and high FRET populations expressing quenched donor. In these experiments unquenched donor lifetimes were around t=2.4 ns while successful quenched populations, exhibiting H-FRET, had lifetimes around 1.15 ns. Experiments were performed on FLS lines transfected with either empty vector or constructs to express WT RPTPα (WT-WT), the P210LP211L mutant (P210-P210 cells) or the dD2 truncation (dD2-dD2 cells), assessing 20-30 cells and four different ako lines per construct across 4-6 experimental repeats.

Consistent with the earlier described techniques through N-STORM and SE FRET and AP FRET, the FLIM study showed that % FRET efficiency of clustered RPTPα was significantly higher in WT-WT compared to either P210-P210 or dD2-dD2 FLS (FIG. 3C).

The above-described experiments indicated that clustered RPTPα recruits SRC to LEDGE of migrating FLS to facilitate stress fiber formation and cell migration. Cortactin is known as a key substrate of SRC. Once phosphorylated, cortactin is recruited to the actin cytoskeleton and contributes to stress fiber stabilization, and cortactin staining serves as a marker for polarized LEDGE formation.

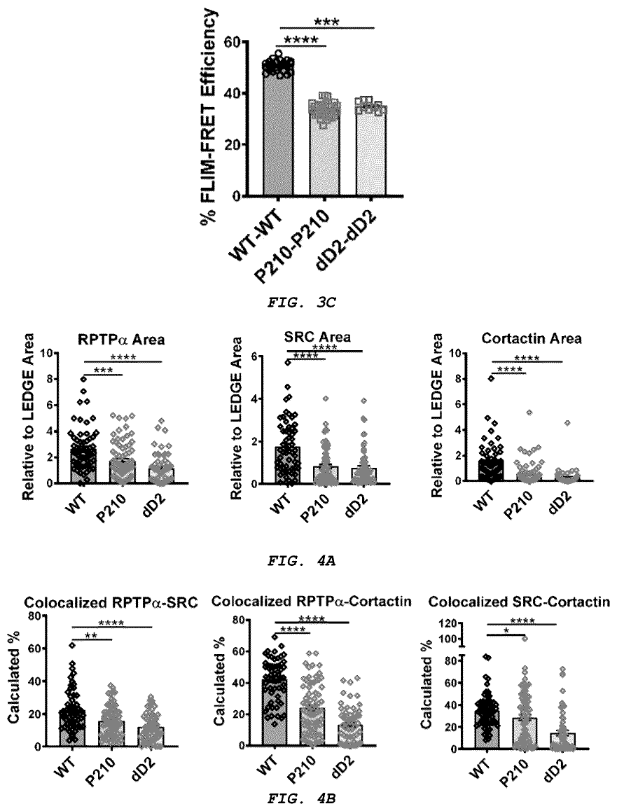

The relationship of RPTPα clustering and RPTPα-SRC association with cortactin recruitment to the actin cytoskeleton in migrating FLS was assessed. Staining of migrating FLS with anti-cortactin Ab showed that cortactin was highly expressed in the LEDGE of WT RPTPα-expressing FLS compared with RPTPα dimerization-deficient mutants, correlating with reduced recruitment of RPTPα and SRC to the same region (FIG. 4A). In transfected cells cortactin co-localized with the areas of RPTPα-SRC FRET. RPTPα-SRC colocalization, RPTPα-cortactin colocalization and SRC-cortactin colocalization was also decreased in the LEDGE of FLS expressing dimerization-impaired mutants of RPTPα (FIG. 4B). These results indicated that RPTPα clustering and RPTPα-SRC association facilitated recruitment of RPTPα, SRC and cortactin to the LEDGE of FLS, which facilitated further F-actin assembly in the polarized lamellipodial leading edge.

### RPTPα Clustering-Deficient Mutations Resulted in Phosphatase Loss of Function in Migration Assays.

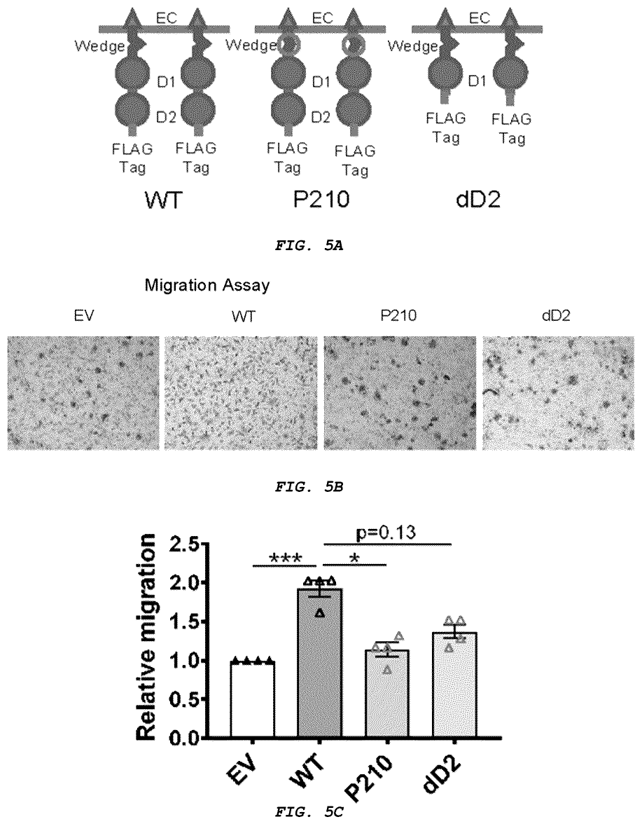

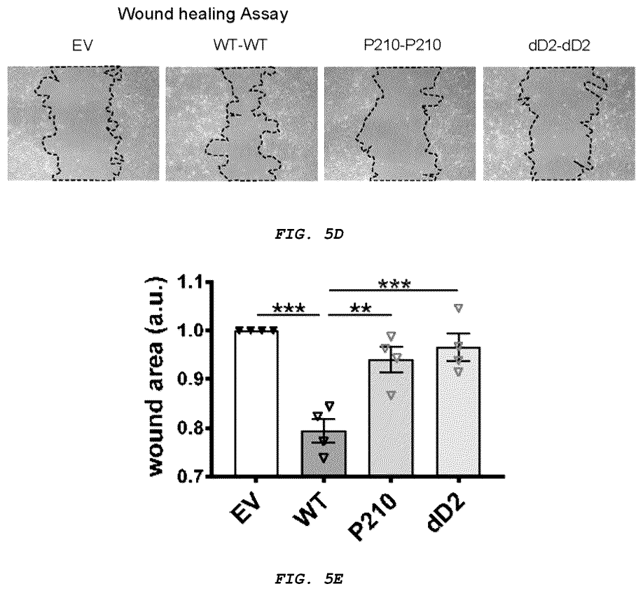

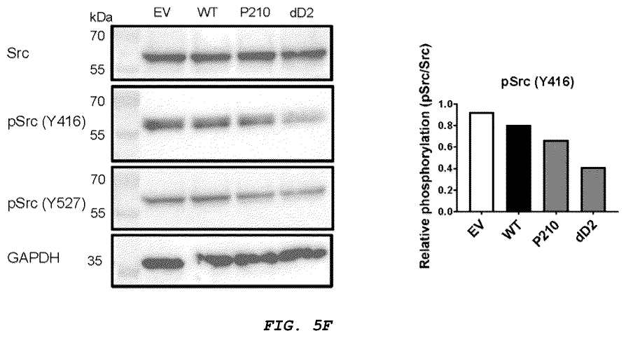

The above-mentioned data indicated that clustering of RPTPα promoted SRC activation in migrating FLS. Since SRC-dependent promotion of F-actin assembly is a key enabler of fibroblast migration, FLS lines transfected with either empty vector or constructs to express WT RPTPα, the P210LP211L or the dD2 truncation mutants in FBS-driven migration assays were assessed (FIG. 5A). Results showed that expression of WT RPTPα, but not the P210LP211L or the dD2 truncation, promoted migration of FLS in two independent assays (FIG. 5B-E), showing that clustering of RPTPα was necessary for promotion of FLS migration.

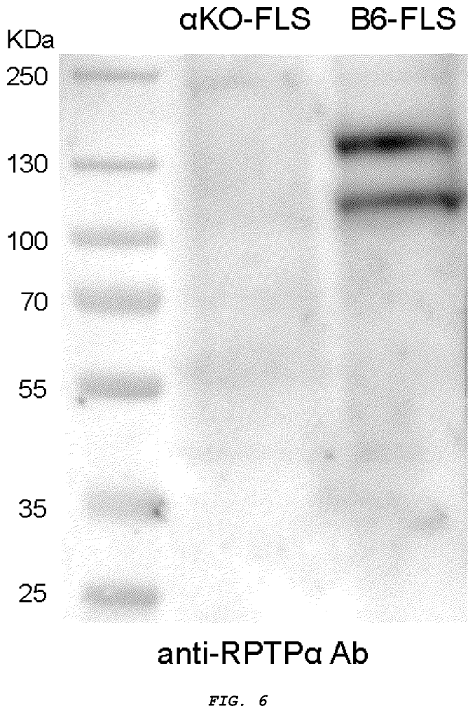

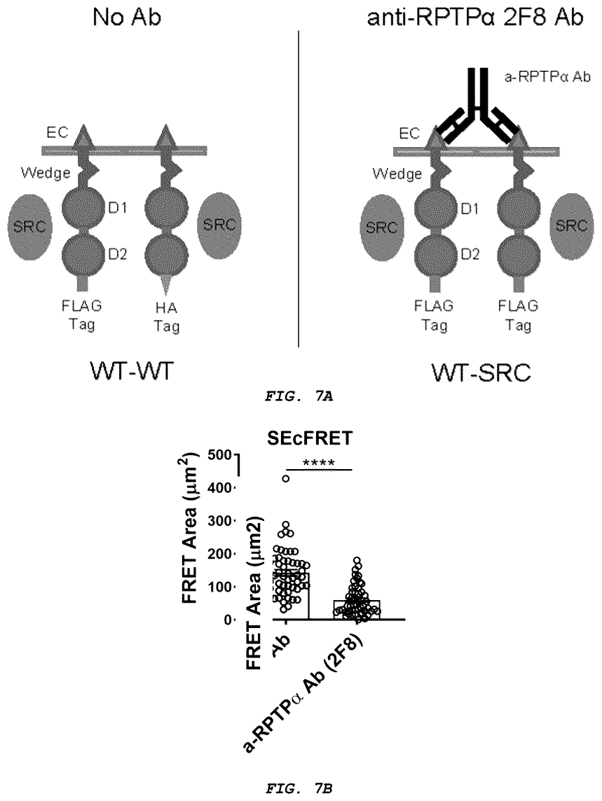

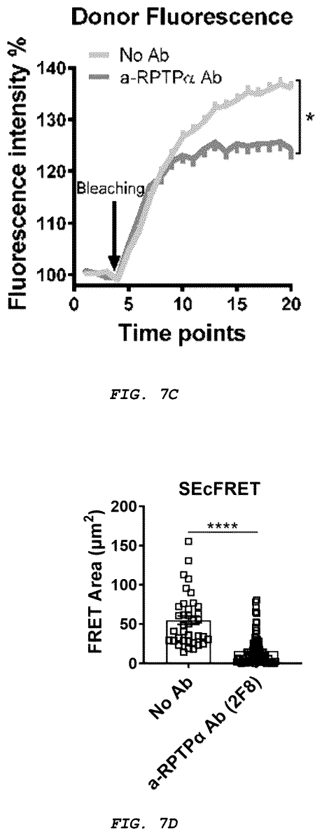

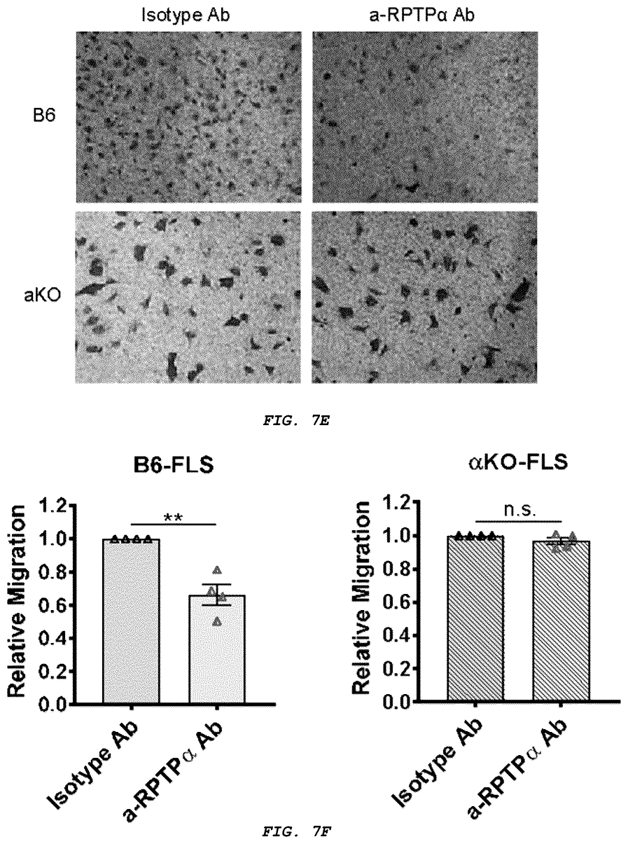

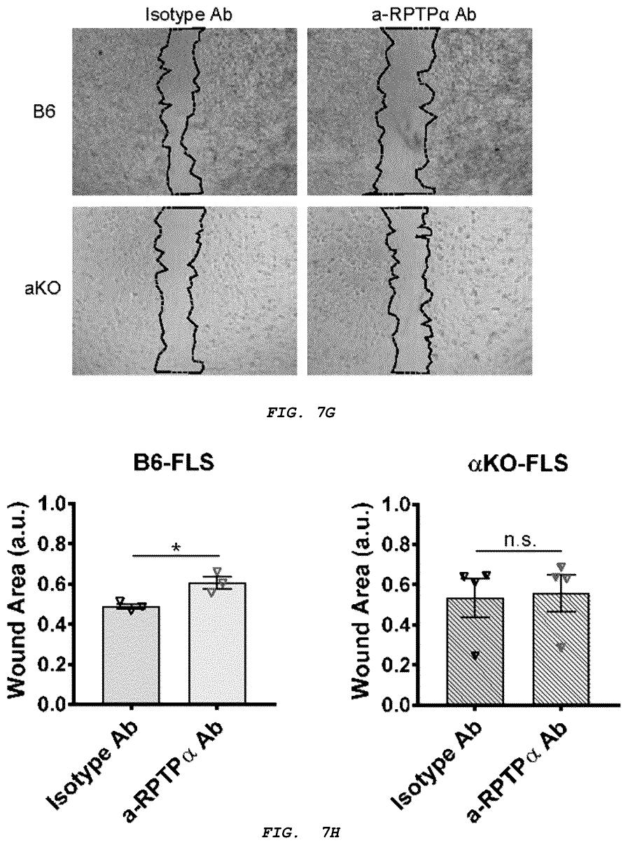

Experiments were performed to acutely alter the clustering of RPTPα through interventions other than mutations looking in parallel for enhanced microscopy phenotypes and RPTPα-dependent changes in WT FLS migration assays. For these experiments, a known monoclonal antibody (clone 2F8) raised against the extracellular domain of RPTPα was used. FIG. 6 shows a Western blot of lysates of B6 and RPTPα KO FLS with the 2F8 antibody to confirm it specifically recognizes RPTPα. First, using αKO FLS transfected with plasmids expressing FLAG-tagged WT RPTPα with or without HA-tagged RPTPα expression constructs (named WT-WT and WT-P210; FIG. 7A), the effects of a known monoclonal antibody (clone 2F8) raised against the extracellular domain of RPTPα on the clustering of RPTPα and its association with SRC in migrating FLS were assessed. Incubation of migrating WT-WT and WT-SRC FLS with 2F8 but not with isotype control Ab or control vehicle led to a mild but significant de-clustering of RPTPα (FIG. 7B-C) and reduced association of RPTPα with SRC (FIG. 7D) in SECFRET and apFRET assays. Results shown in FIG. 7E-H demonstrated that incubation of FLS with 500-1000 nM 2F8 antibody solution lead to a significant reduction of migration of WT but not αKO FLS in Boyden chamber (FIG. 7E-F) or in scratch-wound (FIG. 7G-H) migration assays. Thus, acute de-clustering of endogenous RPTPα by 2F8 correlated with an RPTPα loss of function phenotype in migrating FLS.

### Identification of Epitopes on RPTPα Involved in Clustering.

The region of binding of the 2F8 antibody to RPTPα was mapped using immunoprecipitations and Western blotting of truncation mutants of mouse RPTPα. FLAG-tagged glycosylation-deficient RPTPα truncation mutants (N-terminal truncation mutants termed P1, P2, P3, and P4) were transfected into 293T cells. Anti-FLAG immunoprecipitations were performed on cell lysates, and immunoprecipitates were subjected to Western blotting using 2F8 antibody. Western blotting revealed the 2F8 antibody recognized full-length glycosylation-deficient RPTP and the P1, P2, and P3 mutants, but did not recognize the P4 mutant. These data indicated that 2F8 bound to RPTPα in the region between P3 and P4.

The same FLAG-tagged glycosylation-deficient N-terminal RPTPα truncation mutants described above were transfected into 293T cells. Immunoprecipitations were performed using 2F8 antibody followed by Western blotting using an anti-PTPRA antibody. Western blotting revealed the 2F8 antibody immunoprecipitated the full-length and P3 glycosylation-deficient RPTPα but not the P4 mutant. These data indicated that 2F8 bound to RPTPα in the region between P3 and P4. The amino acid sequence in the region between P3 and P4 truncations in mouse RPTPα is provided in SEQ ID NO:1 (mouse protein sequence, 35 amino acids) and the corresponding region in human RPTPα is provided in SEQ ID NO:2 (human protein sequence, 35 amino acids).

Inhibitors of RPTPα Clustering Slow Cancer Cell Growth and/or Migration.

When cancer cells are exposed to the 2F8 antibody, the growth of these cells is inhibited. The growth of these cells is detected using an established in vitro cell growth assay. Cells are plated into wells of a 12-well plate and allowed to grow for 3-5 days. Cells are then fixed with 70% ethanol and stained with 0.05% crystal violet in 25% ethanol. Plates are then rinsed with water and left to dry overnight. Crystal violet is extracted using Sorenson's extraction reagent (50 mM sodium citrate and 50 mM citric acid dissolved in 50% ethanol). Absorbance of the extracted solution is read at 595 nm using a plate-reader. When cells are treated with 2F8 versus a control antibody, vehicle, or media alone, the growth of the cells is inhibited compared to the control-treated cells.

When cancer cells are exposed to the 2F8 antibody, the migration of these cells is inhibited. The migration of these cells is detected using an established in vitro transwell migration assay. As an example of such an assay, cells are plated in culture media with 0.5% bovine serum albumin into the upper chamber of transwell culture inserts with 8 micron pore size. Inserts are placed into wells containing culture media with 5-10% fetal bovine serum. The cells are allowed to migrate for 4-48 hours, after which the upper chamber of the transwell is wiped to remove non-migrating cells, and the chamber is fixed in methanol and stained with 0.5% crystal violet in 25% methanol. A microscope is used to visualize the chamber and count migrating cells. When cells are treated with 2F8 versus a control antibody, vehicle, or media alone, the migration of the cells is inhibited compared to the control-treated cells.

1. A method of treating an autoimmune disease in a subject in need thereof, the method comprising administering to said subject an effective amount of a protein tyrosine phosphatase receptor type A (RPTPα) antagonist to a subject.

2. The method of claim 1, wherein the RPTPα antagonist reduces the invasiveness or migration of the subject's fibroblast-like synoviocytes.

3. The method of claim 1, wherein said RPTPα antagonist is an anti-RPTPα antibody.

4. The method of claim 3, wherein said anti-RPTPα antibody binds an extracellular portion of RPTPα.

5. The method of claim 3, wherein said anti-RPTPα antibody is an anti-RPTPα dimer inhibiting antibody or an anti-RPTPα declustering antibody.

6. The method of claim 1, wherein said autoimmune disease is arthritis or a fibroblast mediated disease.

7. The method of claim 6, wherein said arthritis is rheumatoid arthritis, psoriatic arthritis, juvenile idiopathic arthritis, or osteoarthritis.

8. The method of claim 1, wherein said autoimmune disease is selected from the group consisting of multiple sclerosis, systemic lupus erythematosus (SLE), myasthenia gravis, juvenile onset diabetes, diabetes mellitus type 1, Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, psoriasis, ichthyosis, Graves ophthalmopathy, inflammatory bowel disease, Addison's disease, Vitiligo, asthma, scleroderma, systemic sclerosis, and allergic asthma.

9. The method of claim 1, wherein said subject comprises fibroblast-like synoviocytes comprising clustered RPTPα and increased RPTPα activity relative to a standard control.

10. A method of treating a disease in a subject, the method comprising administering to the subject a therapeutically effective amount of a recombinant polypeptide, wherein administration treats the disease in the subject, wherein the recombinant polypeptide causes de-clustering of RPTPα, and wherein the disease is selected from the group consisting of an autoimmune disease, an inflammatory autoimmune disease, a fibroblast-mediated disease, or cancer.

11. The method of claim 10, wherein the autoimmune disease is arthritis.

12. The method of claim 10, wherein the autoimmune disease is rheumatoid arthritis.

13. The method of claim 10, wherein the subject has a fibroblast-mediated disease.

14. The method of claim 13, wherein the fibroblast-mediated disease is fibrosis.

15. The method of claim 14, wherein the fibrosis is selected from the group consisting of pulmonary fibrosis, idiopathic pulmonary fibrosis, liver fibrosis, endomyocardial fibrosis, atrial fibrosis, mediastinal fibrosis, myelofibrosis, retroperitoneal fibrosis, nephrogenic systemic fibrosis, skin fibrosis, and arthrofibrosis.

16. The method of claim 10, wherein the disease is cancer.

17. An antibody which binds to one or more epitopes which has or have an amino acid sequence present in SEQ ID NO: 1 or SEQ ID NO:2, or comprising the amino acid sequence of SEQ ID NO: 1 or SEQ ID NO: 2.

18. A pharmaceutical composition comprising the antibody of claim 17 as an active ingredient and a pharmaceutically acceptable carrier.


SEQUENCE LISTING (16 sequences)

SEQ ID NO: 1 - protein, 35 aa, synthetic construct
ESGGTTISPNGSWLIENQFTDAITEPWEGNSSTAA

SEQ ID NO: 2 - protein, 35 aa, synthetic construct
NSIGITISPNGTWLPDNQFTDARTEPWEGNSSTAA

SEQ ID NO: 3 - protein, 35 aa, synthetic construct
NSVGITVSPNGTWLPDNQFTDARTEPWEGNSSTAA

SEQ ID NO: 4 - protein, 35 aa, synthetic construct
NSIGTTILPNETWLPDNQFTDARTEPWEGNSSTAA

SEQ ID NO: 5 - protein, 35 aa, synthetic construct
NSVVTTLSPNGTWLPDNQFTDAGTESWEGNSSTVA

SEQ ID NO: 6 - protein, 35 aa, synthetic construct
NSVVTTISPNGTWLPDNQFTDTRTEPWEGNASTAA

SEQ ID NO: 7 - protein, 38 aa, synthetic construct
ESGGTTISPNGTYGTWLTDNQFTDARTEPWEGNSSTAA

SEQ ID NO: 8 - protein, 46 aa, synthetic construct
STPGTSVPTSAPVAISLPPSATPSALLTALPSTEAEMTERNVSATV

SEQ ID NO: 9 - protein, 35 aa, synthetic construct
DSVVTTISPNGTWLPDDQFTDARTDPWMGNSSTAA

SEQ ID NO: 10 - protein, 151 aa, synthetic construct
MDSWFILVLLGSGLICVSANNATTVAPSVGITRLINSSTAEPVKEEAKTSNPTSSLTSLSVAPTFSPNITLGPTYLTTVNSSDSDNGTTRTASTNSIGITISPNGTWLPDNQFTDARTEPWEGNSSTAATTPETFPPSGNSDSKDRRDETP

SEQ ID NO: 11 - protein, 142 aa, synthetic construct
MDSWFILVLLGSGLICVSANNATTVAPSVGITRLINSSTAEPVKEEAKTSNPTSSLTSLSVAPTFSPNITLGPTYLTTVNSSDSDNGTTRTASTNSIGITISPNGTWLPDNQFTDARTEPWEGNSSTAATTPETFPPSDETP

SEQ ID NO: 12 - protein, 142 aa, synthetic construct
MDSWFILVLFGSGLIHVSANNATTVSPSLGTTRLIKTSTTELAKEENKTSNSTSSVISLSVAPTFSPNLTLEPTYVTTVNSSHSDNGTRRAASTESGGTTISPNGSWLIENQFTDAITEPWEGNSSTAATTPETFPPADETP

SEQ ID NO: 13 - DNA, 2409 bp, Homo sapiens
  source 1..2409: mol_type = other DNA
  CDS <1..>2409: protein_id = 15; translation = MDSWFILVLLGSGLICVSANNATTVAPSVGITRLINSSTAEPVK EEAKTSNPTSSLTSLSVAPTFSPNITLGPTYLTTVNSSDSDNGTTRTASTNSIGITIS PNGTWLPDNQFTDARTEPWEGNSSTAATTPETFPPSGNSDSKDRRDETPIIAVMVALS SLLVIVFIIIVLYMLRFKKYKQAGSHSNSFRLSNGRTEDVEPQSVPLLARSPSTNRKY PPLPVDKLEEEINRRMADDNKLFREEFNALPACPIQATCEAASKEENKEKNRYVNILP YDHSRVHLTPVEGVPDSDYINASFINGYQEKNKFIAAQGPKEETVNDFWRMIWEQNTA TIVMVTNLKERKECKCAQYWPDQGCWTYGNIRVSVEDVTVLVDYTVRKFCIQQVGDMT NRKPQRLITQFHFTSWPDFGVPFTPIGMLKFLKKVKACNPQYAGAIVVHCSAGVGRTG TFVVIDAMLDMMHTERKVDVYGFVSRIRAQRCQMVQTDMQYVFIYQALLEHYLYGDTE LEVTSLETHLQKIYNKIPGTSNNGLEEEFKKLTSIKIQNDKMRTGNLPANMKKNRVLQ IIPYEFNRVIIPVKRGEENTDYVNASFIDGYRQKDSYIASQGPLLHTIEDFWRMIWEW KSCSIVMLTELEERGQEKCAQYWPSDGLVSYGDITVELKKEEECESYTVRDLLVTNTR ENKSRQIRQFHFHGWPEVGIPSDGKGMISIIAAVQKQQQQSGNHPITVHCSAGAGRTG TFCALSTVLERVKAEGILDVFQTVKSLRLQRPHMVQTLEQYEFCYKVVQEYIDAFSDY ANFK
atggattcctggttcattcttgttctgctcggcagtggtctgatatgtgtcagtgccaacaatgctaccacagttgcaccttctgtaggaattacaagattaattaactcatcaacggcagaaccagttaaagaagaggccaaaacttcaaatccaacttcttcactaacttctctttctgtggcaccaacattcagcccaaatataactctgggacccacctatttaaccactgtcaattcttcagactctgacaatgggaccacaagaacagcaagcaccaattctataggcattacaatttcaccaaatggaacgtggcttccagataaccagttcacggatgccagaacagaaccctgggaggggaattccagcaccgcagcaaccactccagaaactttccctccttcaggtaattctgactcgaaggacagaagagatgagacaccaattattgcggtgatggtggccctgtcctctctgctagtgatcgtgtttattatcatagttttgtacatgttaaggtttaagaaatacaagcaagctgggagccattccaattctttccgcttatccaacggccgcactgaggatgtggagccccagagtgtgccacttctggccagatccccaagcaccaacaggaaatacccacccctgcccgtggacaagctggaagaggaaattaaccggagaatggcagacgacaataagctcttcagggaggaattcaacgctctccctgcatgtcctatccaggccacctgtgaggctgcttccaaggaggaaaacaaggaaaaaaatcgatatgtaaacatcttgccttatgaccactctagagtccacctgacaccggttgaaggggttccagattctgattacatcaatgcttcattcatcaacggctaccaagaaaagaacaaattcattgctgcacaaggaccaaaagaagaaacggtgaatgatttctggcggatgatctgggaacaaaacacagccaccatcgtcatggttaccaacctgaaggagagaaaggagtgcaagtgcgcccagtactggccagaccaaggctgctggacctatgggaatattcgggtgtctgtagaggatgtgactgtcctggtggactacacagtacggaagttctgcatccagcaggtgggcgacatgaccaacagaaagccacagcgcctcatcactcagttccactttaccagctggccagactttggggtgccttttaccccgatcggcatgctcaagttcctcaagaaggtgaaggcctgtaaccctcagtatgcaggggccatcgtggtccactgcagtgcaggtgtagggcgtacaggtacctttgtcgtcattgatgccatgctggacatgatgcatacagaacggaaggtggacgtgtatggctttgtgagccggatccgggcacagcgctgccagatggtgcaaaccgatatgcagtatgtcttcatataccaagcccttctggagcattatctctatggagatacagaactggaagtgacctctctagaaacccacctgcagaaaatttacaacaaaatcccagggaccagcaacaatggattagaggaggagtttaagaagttaacatcaatcaaaatccagaatgacaagatgcggactggaaaccttccagccaacatgaagaagaaccgtgttttacagatcattccatatgaattcaacagagtgatcattccagttaagcggggcgaagagaatacagactatgtgaacgcatcctttattgatggctaccggcagaaggactcctatatcgccagccagggccctcttctccacacaattgaggacttctggcgaatgatctgggagtggaaatcctgctctatcgtgatgctaacagaactggaggagagaggccaggagaagtgtgcccagtactggccatctgatggactggtgtcctatggagatattacagtggaactgaagaaggaggaggaatgtgagagctacaccgtccgagacctcctggtcaccaacaccagggagaataagagccggcagatccggcagttccacttccatggctggcctgaagtgggcatccccagtgacggaaagggcatgatcagcatcatcgccgccgtgcagaagcagcagcagcagtcagggaaccaccccatcaccgtgcactgcagcgccggggcaggaaggacggggaccttctgtgccctgagcaccgtcctggagcgtgtgaaagcagaggggattttggatgtcttccagactgtcaagagcctgcggctacagaggccacacatggtccagacactggaacagtatgagttctgctacaaggtggtgcaggagtatattgatgcattctcagattatgccaacttcaagtaa

SEQ ID NO: 14 - DNA, 1611 bp, Homo sapiens
  source 1..1611: mol_type = other DNA
  CDS <1..>1611: protein_id = 16; translation = MGSNKSKPKDASQRRRSLEPAENVHGAGGGAFPASQTPSKPASA DGHRGPSAAFAPAAAEPKLFGGFNSSDTVTSPQRAGPLAGGVTTFVALYDYESRTETD LSFKKGERLQIVNNTEGDWWLAHSLSTGQTGYIPSNYVAPSDSIQAEEWYFGKITRRE SERLLLNAENPRGTFLVRESETTKGAYCLSVSDFDNAKGLNVKHYKIRKLDSGGFYIT SRTQFNSLQQLVAYYSKHADGLCHRLTTVCPTSKPQTQGLAKDAWEIPRESLRLEVKL GQGCFGEVWMGTWNGTTRVAIKTLKPGTMSPEAFLQEAQVMKKLRHEKLVQLYAVVSE EPIYIVTEYMSKGSLLDFLKGETGKYLRLPQLVDMAAQIASGMAYVERMNYVHRDLRA ANILVGENLVCKVADFGLARLIEDNEYTARQGAKFPIKWTAPEAALYGRFTIKSDVWS FGILLTELTTKGRVPYPGMVNREVLDQVERGYRMPCPPECPESLHDLMCQCWRKEPEE RPTFEYLQAFLEDYFTSTEPQYQPGENL
atgggtagcaacaagagcaagcccaaggatgccagccagcggcgccgcagcctggagcccgccgagaacgtgcacggcgctggcgggggcgctttccccgcctcgcagacccccagcaagccagcctcggccgacggccaccgcggccccagcgcggccttcgcccccgcggccgccgagcccaagctgttcggaggcttcaactcctcggacaccgtcacctccccgcagagggcgggcccgctggccggtggagtgaccacctttgtggccctctatgactatgagtctaggacggagacagacctgtccttcaagaaaggcgagcggctccagattgtcaacaacacagagggagactggtggctggcccactcgctcagcacaggacagacaggctacatccccagcaactacgtggcgccctccgactccatccaggctgaggagtggtattttggcaagatcaccagacgggagtcagagcggttactgctcaatgcagagaacccgagagggaccttcctcgtgcgagaaagtgagaccacgaaaggtgcctactgcctctcagtgtctgacttcgacaacgccaagggcctcaacgtgaagcactacaagatccgcaagctggacagcggcggcttctacatcacctcccgcacccagttcaacagcctgcagcagctggtggcctactactccaaacacgccgatggcctgtgccaccgcctcaccaccgtgtgccccacgtccaagccgcagactcagggcctggccaaggatgcctgggagatccctcgggagtcgctgcggctggaggtcaagctgggccagggctgctttggcgaggtgtggatggggacctggaacggtaccaccagggtggccatcaaaaccctgaagcctggcacgatgtctccagaggccttcctgcaggaggcccaggtcatgaagaagctgaggcatgagaagctggtgcagttgtatgctgtggtttcagaggagcccatttacatcgtcacggagtacatgagcaaggggagtttgctggactttctcaagggggagacaggcaagtacctgcggctgcctcagctggtggacatggctgctcagatcgcctcaggcatggcgtacgtggagcggatgaactacgtccaccgggaccttcgtgcagccaacatcctggtgggagagaacctggtgtgcaaagtggcggactttgggctggctcggctcattgaagacaatgagtacacggcgcggcaaggtgccaaattccccatcaagtggacggctccagaagctgccctctatggccgcttcaccatcaagtcggacgtgtggtccttcgggatcctgctgactgagctcaccacaaagggacgggtgccctaccctgggatggtgaaccgcgaggtgctggaccaggtggagcggggctaccggatgccctgcccgccggagtgtcccgagtccctgcacgacctcatgtgccagtgctggcggaaggagcctgaggagcggcccaccttcgagtacctgcaggccttcctggaggactacttcacgtccaccgagccccagtaccagcccggggagaacctctag

SEQ ID NO: 15 - protein, 802 aa, Homo sapiens
MDSWFILVLLGSGLICVSANNATTVAPSVGITRLINSSTAEPVKEEAKTSNPTSSLTSLSVAPTFSPNITLGPTYLTTVNSSDSDNGTTRTASTNSIGITISPNGTWLPDNQFTDARTEPWEGNSSTAATTPETFPPSGNSDSKDRRDETPIIAVMVALSSLLVIVFIIIVLYMLRFKKYKQAGSHSNSFRLSNGRTEDVEPQSVPLLARSPSTNRKYPPLPVDKLEEEINRRMADDNKLFREEFNALPACPIQATCEAASKEENKEKNRYVNILPYDHSRVHLTPVEGVPDSDYINASFINGYQEKNKFIAAQGPKEETVNDFWRMIWEQNTATIVMVTNLKERKECKCAQYWPDQGCWTYGNIRVSVEDVTVLVDYTVRKFCIQQVGDMTNRKPQRLITQFHFTSWPDFGVPFTPIGMLKFLKKVKACNPQYAGAIVVHCSAGVGRTGTFVVIDAMLDMMHTERKVDVYGFVSRIRAQRCQMVQTDMQYVFIYQALLEHYLYGDTELEVTSLETHLQKIYNKIPGTSNNGLEEEFKKLTSIKIQNDKMRTGNLPANMKKNRVLQIIPYEFNRVIIPVKRGEENTDYVNASFIDGYRQKDSYIASQGPLLHTIEDFWRMIWEWKSCSIVMLTELEERGQEKCAQYWPSDGLVSYGDITVELKKEEECESYTVRDLLVTNTRENKSRQIRQFHFHGWPEVGIPSDGKGMISIIAAVQKQQQQSGNHPITVHCSAGAGRTGTFCALSTVLERVKAEGILDVFQTVKSLRLQRPHMVQTLEQYEFCYKVVQEYIDAFSDYANFK

SEQ ID NO: 16 - protein, 536 aa, Homo sapiens
MGSNKSKPKDASQRRRSLEPAENVHGAGGGAFPASQTPSKPASADGHRGPSAAFAPAAAEPKLFGGFNSSDTVTSPQRAGPLAGGVTTFVALYDYESRTETDLSFKKGERLQIVNNTEGDWWLAHSLSTGQTGYIPSNYVAPSDSIQAEEWYFGKITRRESERLLLNAENPRGTFLVRESETTKGAYCLSVSDFDNAKGLNVKHYKIRKLDSGGFYITSRTQFNSLQQLVAYYSKHADGLCHRLTTVCPTSKPQTQGLAKDAWEIPRESLRLEVKLGQGCFGEVWMGTWNGTTRVAIKTLKPGTMSPEAFLQEAQVMKKLRHEKLVQLYAVVSEEPIYIVTEYMSKGSLLDFLKGETGKYLRLPQLVDMAAQIASGMAYVERMNYVHRDLRAANILVGENLVCKVADFGLARLIEDNEYTARQGAKFPIKWTAPEAALYGRFTIKSDVWSFGILLTELTTKGRVPYPGMVNREVLDQVERGYRMPCPPECPESLHDLMCQCWRKEPEERPTFEYLQAFLEDYFTSTEPQYQPGENL